(19) United States
(12) Patent Application Publication
(10) Pub. No.: US 2026/0055393 A1
(43) Pub. Date: Feb. 26, 2026

(54) EXTRACELLULAR VESICLE PROTEIN ASSAY FOR NONINVASIVE DIAGNOSTICS

(71) Applicant: The Regents of the University of California, Oakland, CA (US)

(72) Inventors: Hsian-Rong TSENG, Oakland, CA (US); Yazhen ZHU, Oakland, CA (US); Hyoyong KIM, Oakland, CA (US)

(73) Assignee: The Regents of the University of California, Oakland, CA (US)

(21) Appl. No.: 18/854,316

(22) PCT Filed: Apr. 5, 2023
(86) PCT No.: PCT/US2023/065393

§ 371 Date: Oct. 4, 2024

Related U.S. Application Data
(60) Provisional application No. 63/327,657, filed on Apr. 5, 2022.

(51) Int. Cl. C12N 15/10

(52) U.S. Cl. CPC C12N 15/1065

(57) ABSTRACT

The embodiments of the present invention relate to devices, methods and kits for assaying a disease in a subject, by selectively capturing extracellular vesicles (EVs) by click chemistry with functionalized antibodies, selectively labeling the EVs with a plurality of DNA barcodes conjugated antibodies, optionally releasing the plurality of DNA barcodes, and assaying the plurality of DNA barcodes to determine whether the disease is present or predict the stage of the disease in the subject.

### CROSS-REFERENCE OF RELATED APPLICATIONS

This application is a U.S. National Stage of PCT/US2023/065393 filed Apr. 5, 2023, which claims priority to U.S. Provisional Application No. 63/327,657 filed Apr. 5, 2022; the entire contents of which are hereby incorporated by reference.

### GOVERNMENT SUPPORT

This invention was made with government support under Grant Numbers CA240887, EB026421, CA255727, and CA253651, awarded by the National Institutes of Health. The government has certain rights in the invention.

### SEQUENCE LISTING

The instant application contains a Sequence Listing XML which has been submitted electronically and is hereby incorporated by reference in its entirety. Said XML copy, created on Apr. 5, 2023, is named 58086-575390 and is 11,174 bytes in size.

### BACKGROUND

### 1. Technical Field

The currently claimed embodiments of the present invention relate to devices, methods and kits for assaying a disease in a subject, and more particularly to devices, methods and kits for assaying a disease in a subject by selectively labeling, capturing, and quantifying extracellular vesicles (EVs).

### 2. Discussion of Related Art

Primary liver cancer is the third-leading cause of cancer-related death worldwide in 2020.¹ In the United States, despite the plateaued incidence and mortality trends over the past decade,^{2,3} more than 30,000 deaths from primary liver cancer are estimated in 2021.⁴ Hepatocellular carcinoma (HCC) accounts for 80-85% of primary liver cancers^{5,6} and mainly occurs in patients with liver cirrhosis or chronic hepatitis B virus (HBV) infection.⁶ Prognosis of HCC is dismal primarily due to advanced stage of disease at diagnosis, and studies have shown early detection of HCC is associated with increased receipt of curative therapy and improved patient prognosis.^{3,7} Current clinical practice guidelines recommend HCC surveillance by biannual liver ultrasound with/without serum alpha-fetoprotein (AFP) for at-risk patients to achieve the goal of detecting HCC at a curative stage.^{8,9} However their accuracy remains relevantly low with sensitivity between 60-70% at a specificity of 90%.¹⁰⁻¹³ As such, novel biomarkers for early detection of HCC are still desperately needed. Extracellular vesicles (EVs) refer to nanoparticles released by both normal and tumor cells.^{14,15} Enclosed by lipid bilayer membranes, EVs contain biomolecules including DNA, RNA, proteins, metabolites, and lipids. Through transferring these cargoes, tumor-derived EVs participate in cell-to-cell communications and cancer progression. Given their early presence in circulation during tumorigenesis, profiling tumor-derived EVs is regarded as a promising liquid biopsy approach for diagnosis of early-stage cancer.^{14,15} There have been several studies investigating the role of EVs in detecting HCC among at-risk patients with highly promising results.^{14,16,17} For example, von Felden et al. and Tseng et al demonstrated the noncoding RNA¹⁶ and mRNA¹⁷ markers in EVs can be used to discriminate Barcelona Clinic liver cancer (BCLC) stage 0-A HCC from cirrhosis with area under receiver operator characteristic curves (AUROCs) of 0.93, respectively. Therefore, EVs hold great potential to augment current surveillance strategy and improve HCC patients' outcomes.

Given that the sensitivity of current surveillance methods for detecting early-stage hepatocellular carcinoma (HCC) is suboptimal. Extracellular vesicles (EVs) are promising circulating biomarkers for early cancer detection. In this study, we aim to develop an HCC EV-based surface protein assay for early detection of HCC.

### INCORPORATION BY REFERENCE

All publications and patent applications identified herein are incorporated by reference in their entirety and to the same extent as if each individual publication or patent application was specifically and individually indicated to be incorporated by reference.

### BRIEF DESCRIPTION OF THE DRAWINGS

FIGS. 1A-ID provides a schematic of the workflow of an embodiment with EV Click Beads for isolation and surface protein characterization of hepatocellular carcinoma extracellular vesicles (HCC EVs). (A) Schematic illustration of the work mechanism of EV Click Beads, which are covalently functionalized with tetrazine (Tz); the click chemistry-mediated EV capture is combined with the use of antibodies targeting HCC-associated surface markers, i.e., EpCAM, ASGPR, GPC3 and CD147 (FIG. 1B). A pair of highly reactive click chemistry motifs, i.e., Tz and trans-cyclooctene (TCO), are grafted onto Click Beads and EVs, respectively. When a plasma sample goes through the workflow, click chemistry reaction between Tz-grafted Click Beads and TCO-grafted HCC EVs results in the immobilization of the HCC EVs onto the Click Beads (FIG. 1C). Subsequently, the captured HCC EVs were quantified by duplexed Realtime PCR to detect the DNA barcodes conjugated with CD63 or CD9 (FIG. 1D). The resulting signatures of HCC EV surface protein markers will be analyzed for differentiating HCC versus liver cirrhosis.

FIGS. 2A-2B is a schematic illustration of workflow for HCC EV SPA; (A) An embodiment of antibody-DNA barcoding for HCC EV SPA and (B) An embodiment of a library preparation and NGS analysis of DNA barcode.

FIGS. 3A-3B is a schematic illustration of workflow for HCC EV SPA; (A) An embodiment of antibody-DNA barcoding for HCC EV SPA and (B) An embodiment of a NanoString system-based DNA barcode analysis.

FIG. 4 describes an embodiment for the preparation of the click beads.

FIG. 5 describes an embodiment for the preparation of four TCO-conjugated HCC-specific antibodies.

FIGS. 6A-6E describe a process for the conjugation of DNA barcode onto antibody and validation of the conjugated DNA-antibody.

FIG. 7 provides a table of DNA barcode sequences SEQ ID NO: 1-2 and probes/primers SEQ ID NO: 3-8 for the duplex real-time PCR.

FIGS. 8A-8C describe the characterization of HCC cell line-derived EVs. (A) Concentration and size distributions of HepG2-derived (B) Expression of EV markers (CD63 and Annexin V) on HepG2-derived EVs was analyzed by flow cytometry. (C) Representative TEM image of HepG2-derived EVs in bulk solution before capture with immunogold labeling.

FIGS. 9A-9F provide data on optimization of isolation of HCC EV by Click Beads.

FIG. 10 describe the characterization of HepG2-derived EVs captured on Click Beads by EM.

FIG. 11 illustrates an embodiment of a workflow of HCC EV Surface Protein Assay for quantification of eight subpopulations of HCC EVs.

FIGS. 12A-12H provide the optimization of HCC EV Surface Protein Assay using HCC cell line-derived EVs.

FIG. 13 shows Validation of reproducibility of HCC EV Surface Protein Assay using clinical plasma samples.

FIG. 14 shows ROC curve of the eight HCC EV subpopulations for detecting early-stage HCC from cirrhosis in the UCLA training cohort.

FIG. 15 shows Table 2 presenting the relative signal for evaluation of reproducibility of HCC EV Surface Protein Assay.

FIG. 16 shows Table 3 presenting univariate logistic regression analysis of the eight HCC subpopulations for detecting early-stage HCC from cirrhosis.

FIG. 17 illustrates a Flow chart describing one embodiment.

FIG. 18 show the clinical study design flowchart depicting the recruitment and exclusions from the study.

FIGS. 19A-19D show the validation of the four selected HCC-associated surface markers using tissue microarray (TMA) and HCC cells-derived EVs.

FIGS. 20A-20E show HCC EV Surface Protein Assay (SPA) for measuring subpopulations of HCC EVs and detecting early-stage HCC in the UCLA training cohort.

FIGS. 21A-21C show HCC EV ECG score for detecting early-stage HCC in the CSMC independent validation cohort.

FIGS. 22A-22D show comparison between HCC EV ECG score and serum AFP for detecting early-stage HCC.

### SUMMARY

A method of assaying for a disease in a subject, includes: selectively labeling an extracellular vesicle (EV) from a sample from said subject with a first DNA barcode comprising: functionalizing a first capture agent for EV with a first molecule from a first bioorthogonal functional group such that the first capture agent remains attachable to EV and the first molecule is able to ligate with a second molecule from a second bioorthogonal functional group, the second molecule being complementary to the first molecule, functionalizing the DNA barcode with a second molecule, conjugating the functionalized first capture agent with the functionalized DNA barcode to form a barcoded capture agent, mixing the barcoded capture agent with the sample to form a labeled sample; selectively capturing said EV from said sample from said subject includes: functionalizing a second capture agent for said EV with a third molecule from a third bioorthogonal functional group such that the second capture agent remains attachable to said EV and the third molecule is also able to bond to a fourth molecule from a fourth bioorthogonal functional group, the fourth molecule being complementary to the third molecule, mixing the functionalized second capture agent with the labeled sample such that the functionalized second capture agent binds to said EV forming an activated sample, functionalizing a capture surface with the fourth molecule, and depositing at least a portion of the activated sample on at least a portion of the functionalized capture surface to thereby selectively capture said EV by binding of the fourth molecule with the third molecule; assaying the first DNA barcode by a nucleic acid test; and determining from assaying of the first DNA barcode from said EV whether said disease is present in said subject.

A method of assaying for a disease in a subject, includes: selectively labeling an extracellular vesicle (EV) from a sample from said subject with a first DNA barcode includes: functionalizing a first capture agent for EV with a first molecule from a first bioorthogonal functional group such that the first capture agent remains attachable to EV and the first molecule is able to ligate with a second molecule from a second bioorthogonal functional group, the second molecule being complementary to the first molecule, functionalizing the DNA barcode with a second molecule, conjugating the functionalized first capture agent with the functionalized DNA barcode to form a barcoded capture agent; functionalizing a second capture agent for said EV with a third molecule from a third bioorthogonal functional group such that the second capture agent remains attachable to said EV and the third molecule is able to ligate with a fourth molecule from a fourth bioorthogonal functional group, the fourth molecule being complementary to the third molecule, mixing the barcoded capture agent and the functionalized second capture agent with said sample to form an activated sample; selectively capturing said EV from said sample from said subject includes: functionalizing a capture surface with the fourth molecule, and depositing at least a portion of the activated sample on at least a portion of the functionalized capture surface to thereby selectively capture said EV by binding of the fourth molecule with the third molecule; assaying the first DNA barcode by a nucleic acid test; and determining from assaying of the first DNA barcode from said EV whether said disease is present in said subject.

The method of assaying according to any of the preceding embodiments where the second capture agent selected for capturing the EV is a tumor-specific capture agent and binds to a surface marker specifically expressed by a tumor-derived EV.

The method of assaying of any of the preceding embodiments where the selectively capturing of an extracellular vesicle (EV) from a sample from a subject is performed prior to the selectively labeling of said EV with a first DNA barcode from said sample from said subject.

The method of assaying according to the any of the preceding embodiments, further includes: repeating the selectively labeling of the extracellular vesicle (EV) from the sample with a second DNA barcode such that the EV is labeled with a distinct first capture agent that binds to a distinct region of the EV, the first and second DNA barcodes being distinguishable from each other; assaying the second DNA barcode using a nucleic acid test; and determining from assaying of the first and second DNA barcodes from the EV whether the disease is present in the subject.

The method of assaying according to the preceding embodiment, further includes: repeating the selectively labeling of the extracellular vesicle (EV) from the sample with a further plurality of DNA barcodes such that the EV is labeled with a plurality of distinct capture agents each binding to a distinct region of the EV, the first and further plurality of DNA barcodes each being distinguishable from each other; assaying the further plurality of DNA barcodes using a multiplex nucleic acid test; and determining a surface protein expression signature from assaying of the plurality of DNA barcodes.

The method of assaying according to the preceding embodiment further includes: repeating, a plurality of times, the selectively labeling, the selectively capturing, the assaying the DNA barcodes, and the determining of a surface protein expression signature to predict a disease stage and/or distinguish between different disease stages. Each selectively capturing uses a capture agent that binds to a portion of the EV that is different from other capture agents used for the selectively capturing.

The method of assaying according to any of the preceding embodiments further includes: releasing the DNA barcode(s) from the captured EVs before the assaying.

The method of assaying according to any of the preceding embodiments is performed by selectively capturing of an extracellular vesicle (EV) from a sample from a subject prior to selectively labeling of said EV with a plurality of DNA barcodes from said sample from said subject.

The method of assaying according to the preceding embodiment where surface protein expression signatures distinguish between distinct disease stages.

The method of assaying according to the preceding embodiment where the expression of EpCAM+CD63+ on HCC EVs, CD147+CD63+ on HCC EVs, and GPC3+CD63+ on HCC EVs is more elevated in patients with Hepathocarcinoma (HCC) compared to those with cirrhosis.

The method of assaying according to any of the preceding embodiments where the capture agent that enables the capture of the EV is functionalized with a third bioorthogonal functional group selected from the list consisting of trans-cyclooctene (TCO), alkyne, and a cyclooctyne derivative, and where the capture surface is functionalized with a fourth bioorthogonal functional group selected from the list consisting of tetrazine (Tz) and azide.

The method of assaying according to any preceding embodiments where the capture surface is a surface of a bead in a suspension.

The method of assaying according to any preceding embodiments where the capture surface is a surface of a microfluidic device.

The method of assaying according to any preceding embodiments where the assaying comprises using a multiplex PCR.

The method of assaying according to any preceding embodiments where the assaying comprises using a next generation sequencing process.

A kit for assaying for a disease in a subject according to any of the preceding embodiment, includes: a plurality of capture agents each of which bind to a distinct region of an EV; a plurality of molecules having complementary bioorthogonal functional groups; a plurality of DNA barcodes; and instructions for labeling the EV with said plurality of DNA barcodes, capturing the EV, assaying said plurality of DNA barcodes, and determining from assaying whether the disease is present or predict the disease stage in the subject.

The kit according to the preceding embodiment further includes instructions for the releasing of said plurality of DNA barcodes before the assaying.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed without departing from the broad concepts of the present invention. All references cited anywhere in this specification are incorporated by reference as if each had been individually incorporated.

The term “bioorthogonal chemistry” refers to any chemical reaction between azide and alkyne functional groups that are largely inert, or bioorthogonal, towards biological molecules and aqueous environments, and as such do not interfere with native biochemical processes. In some embodiments, a molecule containing a highly reactive azide group is reacted with a strained alkyne containing molecule. The azide and alkyne ‘click’ together and ligate both molecules via a newly formed triazole group. The reaction may or may not require a catalyst such as copper, light or electricity.

A non-limiting example of a bioorthogonal reaction for use according to embodiments of the invention is the 1,3-dipolar cycloaddition between an azide containing molecule and a cyclooctyne as described in the scheme below:

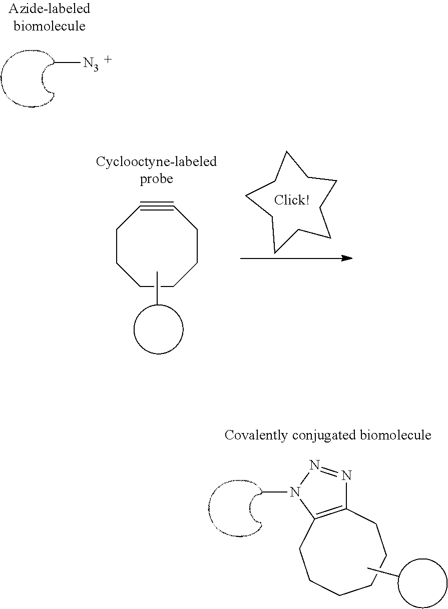

The term DNA barcode as use herein is intended to mean a known DNA sequence that is bioorthogonal to the subject so as to be clearly distinguishable from the genome of the subject. The term “barcodes” refers to unique sequences that can be used to distinguish nucleic acids, even after the nucleic acids are pooled together. In some embodiments, the DNA barcodes may be functionalized and conjugated to capture agents.

Current HCC surveillance is based on ultrasound with/without AFP, despite their suboptimal diagnostic performance. A statistical model, named GALAD score, derived from gender, age, and three serum biomarkers (i.e., AFP-L3, AFP, and Des-carboxyprothrombin), demonstrated high sensitivity in detecting HCC in phase II biomarker studies.26,27 However, the more recent phase III study showed overall lower accuracy, emphasizing the need for better biomarker for HCC surveillance.28 Liquid biopsy has emerged as another promising strategy for HCC surveillance.14,29,30 For example, several circulating tumor DNA-based assays measuring the molecular characteristics have been proposed for detecting HCC and received Food and Drug Administration breakthrough device designation.31-33 In addition, as shown in previous studies, the biomolecular cargoes such as miRNA,14 noncoding RNA,16 mRNA,17 and proteins in EVs are also regarded as potential biomarkers for early detection of HCC.

### Functionalization of the Capture Surface

Some embodiments of the invention include a capture surface for capturing an extracellular vesicle. Further non-limiting examples of capture surface is a surface of a bead in a suspension or may comprise a plurality of Silicon nanostructures. Such capture surface can be functionalized by a molecule having a bioorthogonal functional group that is complementary to a bioorthogonal functional group of a functionalized capture agent. (FIG. 4) In one aspect of the embodiments, beads suitable to covalently capture terminal azide/alkyne-conjugated molecules are used to capture extracellular vesicles (EVs) from a sample.

The shape of the capture surface is not critical. For example, the sphere or bead may be grafted using silane chemistry with at least one molecule having a bioorthogonal functional group able to ligate with a complementary bioorthogonal functional group of another molecule, the other molecule being able to functionalize a capture agent. In other embodiments, the capture surface may comprise a plurality of nanostructures such as nanowires, nanofibers, nanotubes, nano-pillars, nanospheres, or nanoparticles.

As a sample is placed on the functionalized capture surface, extracellular vesicles grafted by at least one capture agent are selectively captured thanks to click chemistry reactions. The capture agent or agents employed will depend on the type of extracellular vesicles being targeted. Conventional capture agents are suitable for use in some of the embodiments of the present invention. Non-limiting examples of capture agents include antibodies, nucleic acids, oligo- or polypeptides, cellular receptors, ligands, aptamers, biotin, avidin, coordination complexes, synthetic polymers, carbohydrates, lipid derivatives, and combination thereof.

In other embodiments, the capture surface is coated with streptavidin and the capture agents are biotinylated to facilitate the immobilization of the EVs.

A capture surface of the embodiments may be overlaid by a microfluidic chaotic mixer allowing a configuration of the throughput flow such as to enhance the physical contact between the sample and at least a portion of the functionalized capture surface. A non-limiting example of a microfluidic chaotic mixer includes a polydimethylsiloxane (PDMS) chaotic mixer.

Some embodiments of the current invention are directed to a method of assaying for a disease in a subject. The subject can be a human or an animal, for example. The method includes selectively labeling an extracellular vesicle (EV) from a sample from the subject with a first DNA barcode, selectively capturing the EV from the sample, optionally releasing the first DNA barcode from the captured EV, assaying a nucleic acid sequence from the first DNA barcode, and determining from the assaying of the first DNA bar ode from the EV whether the disease is present in the subject.

### Selection of Capture Agents

The selection of capture agents either for EV labeling or EV capture merely depends on the disease being diagnosed or evaluated. In some embodiments, the capture agents are selected in order to achieve the desired sensitivity and specificity of recognizing and labeling and/or capturing tumor-derived EVs. In one aspect of the embodiments, the capture agent is an antibody which specifically binds to a tumor-associated surface marker present on the tumor-derived EVs. Non-limited examples of surface markers include antigens, polypeptides, receptors or surface proteins. In one aspect of the embodiments, an antibody may be selected because of its specificity to a tumor-associated surface marker which itself is differentially expression between tumor-derived EVs compared to EVs derived from normal cells or between tumor-derived EVs at different stages of the disease. The differential expression refers to a surface marker being highly expressed (up-regulated), under expressed (down-regulated), expressed restrictively or not expressed in tumor-associated EVs. In another aspect of the embodiments, the markers may preferentially be selected because of their lack of expression from EVs derived from non-disease cells. For example, in a method for capturing EVs derived from a tumor such as HCC, multiple antibodies may be selected because they bind to highly expressed surface markers on HCC-EVs, HCC-CTCs, HCC cell lines, and primary tumor tissues of HCC patients, while not being expressed on white blood cells. For example, in a method for labeling EVs derived for a tumor such as HCC, multiple antibodies may be selected because they bind surface markers expressed on EVs, not necessarily derived from the tumor. Non-limited examples of HCC markers include EpCAM, ASGPR, CD147, and GPC-3. Non-limited examples of HCC EVs capture agents include antibodies anti-EpCAM, anti-ASGPR, anti-CD147, and anti-GPC-3. Non-limited examples of EVs capture agents include antibodies anti-CD63 and anti-CD9.

Tumor heterogeneity includes spatial intratumor heterogeneity within each tumor, and interpatient heterogeneity with genetic and molecular diversity among tumors from different patients. Similar to the parental tumor cells, tumor-derived EVs are highly heterogeneous, underscoring the need for integration of different surface protein markers for developing liquid biopsy-based diagnostic assay to detect early-stage cancer including HCC. HCC tissue microarray showed that four surface protein markers (i.e., EpCAM, CD147, GPC3, and ASGPR1) are complementarily expressed on the tumor cell surfaces. The complementary expression pattern of surface protein markers integrates different subpopulations of tumor-derived EVs for detecting highly heterogeneous tumors. For example, surface protein markers may be included to the scoring with the exception of ASGPR1; although ASGPR1 shows strong positivity in HCC TMA. It's well known that ASGPR1 is a liver-specific marker also expressed on liver cells of liver cirrhotic patients.

In some embodiments, the EVs are labeled by more than one capture agents to optimize the specificity and sensitivity of the method. Considering the fact that EVs can be secreted by highly heterogeneous cell populations, it is conceivable that EVs derived from the same tumor may expressed different surface markers. In one aspect of the embodiments, a plurality of capture agents may be necessary to label EVs, allowing for sensitive and specific detection of tumor-derived EVs across all disease stages. For a given clinical sample, the expression of the plurality of labeling capture agents may be evaluated, thereby providing an expression signature that can be characteristic of a disease stage. Integrating click chemistry-mediated EV capture and a plurality of capture agents such as antibodies offers a more sensitive and specific method for labeling, capturing and assaying tumor-derived EVs with a minimal level of background.

### Affinity Capture: Click Chemistry

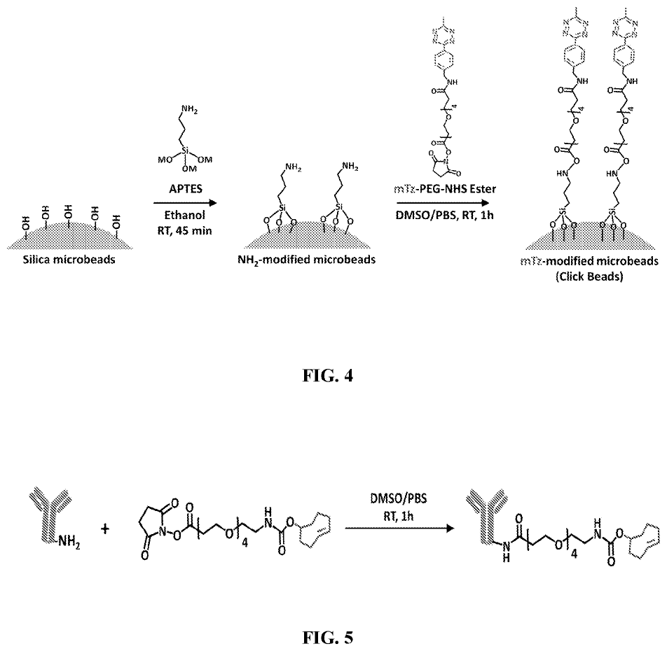

In some embodiments, the capture surface is functionalized with a molecule comprising a bioorthogonal functional group. A non-limiting example of a bioorthogonal functional group includes an azide motif, preferably a Tz motif. The capture agent which immobilized the EV on the surface is functionalized with a molecule having the bioorthogonal functional group selected from the list consisting of trans-cyclooctene (TCO), Alkyne, and a cyclooctyne derivative. (FIG. 5) In one aspect of the embodiments, the cyclooctyne derivative includes dibenzylcyclooctyne (DBCO) or biarylazacyclooctynone (BARAC). In one aspect of the embodiments, a pair of highly reactive click chemistry motifs includes but not limiting Tz and TCO, are grafted onto the capture substrate and capture agent, respectively. In one aspect of the embodiments, the inverse-electron-demand Diels-Alder cycloaddition between Tz and TCO motifs (a rate constant of 10⁴ M-1 s⁻¹) may be selected given their balanced chemical properties concerning both stability and reactivity, without the presence of a catalyst. The ligation between Tz-grafted capture surface and TCO-grafted capture agent is insensitive to biomolecules, water, and oxygen, leading to specific, rapid, and irreversible immobilization of the EVs with improved capture efficiency and reduced nonspecific trapping of particles in the background. Such approach allows for more effective conjugation of the TCO-grafted antibody onto the majority of EVs in a small volume of solution, facilitating the click-chemistry-mediated EV capture onto Click Beads.

In some embodiments, the capture agent that enables the capture of the EV is functionalized with a third bioorthogonal functional group selected from the list consisting of trans-cyclooctene (TCO), alkyne, and a cyclooctyne derivative, and wherein the capture surface is functionalized with a fourth bioorthogonal functional group selected from the list consisting of tetrazine (Tz) and azide. In one aspect of the embodiments, the third molecule having the third bioorthogonal functional group and the fourth molecule having the fourth bioorthogonal functional group are present in a molar ratio of between 2:1 to 10:1. (FIGS. 4 and 5) In one aspect of the embodiment the capture agent that enables the capture of the EV is directed to a surface marker specifically expressed by a tumor-derived EV.

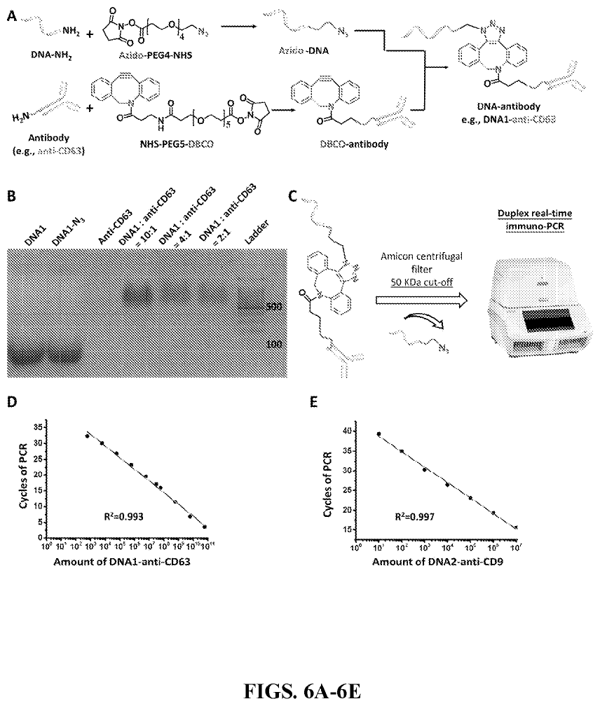

In some embodiments, a capture agent is functionalized with a molecule having a bioorthogonal functional group which is able to ligate with a complementary bioorthogonal functional group of a functionalized DNA barcode, while enabling its binding to its surface marker on the EV bilayer membrane. (FIG. 6). In one aspect of the embodiments, conjugation of capture agent and DNA barcodes is performed with a molar ratio of between 1:2 and 1:10.

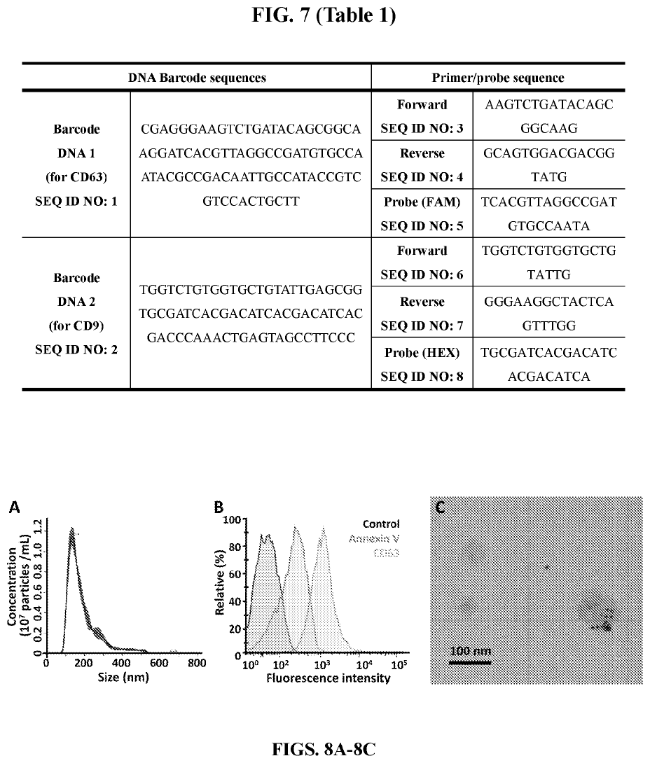

In some embodiments, DNA barcodes are conjugated onto capture agents for targeting EV markers. For example, DNA barcode 1 (SEQ ID NO: 1) and DNA barcode 2 (SEQ ID NO: 2) were conjugated onto antibodies targeting CD63 and CD9 (FIGS. 6A and 7). In some aspect of the embodiments, DNA barcodes are functionalized with a molecule having an azide motif (e.g., azido-PEG₄-NHS) in the molar ratio of 1:20. In another aspect of the embodiments, the capture agents are functionalized with a molecule comprising a complementary biorthogonal functional group to the azide motif. A non-limiting example of complementary biorthogonal functional group is DBCO (diarylcyclooctyne). The functionalization of the capture agent is performed with a molar ratio of between 1:2 and 1:20, preferably 1:10.

In some embodiments, a cleaving motif is incorporated between each pair of DNA barcode and capture agent, allowing for the cleavage and recovery of DNA barcodes for subsequent quantification by multiplex PCR or NGS or any other nucleic acid tests. Non-limiting examples of cleaving motif are disulfide motifs cleavable with DTT or UV irradiation-triggered linkers cleavable by UV irradiation.

In some embodiments, other affinity capture methods can be used, e.g., but not limited to, biotin-streptavidin.

### Selectively Labeling EV

An embodiment is related to a method of assaying for a disease in a subject, includes: selectively labeling an extracellular vesicle (EV) from a sample from said subject with a first DNA barcode comprising: functionalizing a first capture agent for EV with a first molecule from a first bioorthogonal functional group such that the first capture agent remains attachable to EV and the first molecule is able to ligate with a second molecule from a second bioorthogonal functional group, the second molecule being complementary to the first molecule, functionalizing the DNA barcode with a second molecule, conjugating the functionalized first capture agent with the functionalized DNA barcode to form a barcoded capture agent, mixing the barcoded capture agent with the sample to form a labeled sample.

An embodiment is related to a method of assaying for a disease in a subject, includes: selectively labeling an extracellular vesicle (EV) from a sample from said subject with a first DNA barcode includes: functionalizing a first capture agent for EV with a first molecule from a first bioorthogonal functional group such that the first capture agent remains attachable to EV and the first molecule is able to ligate with a second molecule from a second bioorthogonal functional group, the second molecule being complementary to the first molecule, functionalizing the DNA barcode with a second molecule, conjugating the functionalized first capture agent with the functionalized DNA barcode to form a barcoded capture agent, functionalizing a second capture agent for said EV with a third molecule from a third bioorthogonal functional group such that the second capture agent remains attachable to said EV and the third molecule is able to ligate with a fourth molecule from a fourth bioorthogonal functional group, the fourth molecule being complementary to the third molecule, mixing the barcoded capture agent and the functionalized tumor-specific capture agent with said sample to form an activated sample,

In some embodiments, the first capture agent is an antibody which binds to a surface marker specifically expressed on EV but not necessarily an antibody which binds to a surface marker specifically expressed by a tumor-derived EV. When assaying HCC EVs, non-limiting examples includes anti-CD63 and anti-CD9.

In some embodiments, the method includes the simultaneous labeling of the EVs with a functionalized second/tumor-specific capture agent and at least one DNA-capture agent conjugate. In other embodiments, the method includes a sequential labeling of the EVs with a functionalized second/tumor-specific capture agent followed by the labeling of at least one DNA-capture agent conjugate. In other embodiments, the method includes a sequential labeling of the EVs with at least one DNA-capture agent conjugate followed by a functionalized second/tumor-specific capture agent.

In some embodiments, the second capture agent is an antibody which binds to a surface marker specifically expressed on a tumor-derived EV.

### Selectively Capturing EV

In some embodiments, the selectively capturing of an extracellular vesicle (EV) from a sample from a subject is performed prior to the selectively labeling of said EV with one of a plurality of DNA barcodes from said sample from said subject.

An embodiment of a method of selectively capturing an extracellular vesicle (EV) from a sample from a subject further includes: functionalizing a second capture agent for said EV with a third molecule from a third bioorthogonal functional group such that second capture agent remains attachable to said EV and said third molecule is able to ligate with a fourth molecule from a fourth bioorthogonal functional group, the fourth molecule being complementary to the third molecule, mixing the functionalized second capture agent with said sample such that said functionalized second capture agent binds to said EV forming an activated sample, functionalizing a capture surface with the fourth molecule, and depositing at least a portion of the activated sample on at least a portion of the functionalized capture surface to thereby selectively capture said EV by binding of the fourth molecule with the third molecule; assaying said first DNA barcode by nucleic acid test; and determining from assaying of said first DNA barcode from said EV whether said disease is present in said sample.

An embodiment of a method of selectively capturing an extracellular vesicle (EV) from a sample from a subject further includes: functionalizing a capture surface with the fourth molecule, and depositing at least a portion of the activated sample on at least a portion of the functionalized capture surface to thereby selectively capture said EV by binding of the fourth molecule with the third molecule; assaying said first DNA barcode by nucleic acid test; and determining from assaying of said first DNA barcode from said EV whether said disease is present in said subject.

In some aspect of the embodiments, the releasing of said first DNA barcode from said captured EV is performed before assaying said first DNA barcode.

In one aspect of the embodiments, the second capture agent selected for capturing said EV is a tumor-specific capture agent that binds to a surface marker specifically expressed by a tumor-derived EV.

### Surface Protein Expression Signature to Predict the Stage of a Disease

In some embodiments, the EV is selectively labeled with 2 antibodies, each antibody being conjugated with a distinguishable DNA barcode. In another aspect of the embodiments, the method of assaying further includes: repeating said selectively labeling said extracellular vesicle (EV) from said sample with a second DNA barcode such that said EV is labeled with a distinct capture agent binding to a distinct region of an EV, said first and second DNA barcodes being distinguishable from each other; optionally releasing said second DNA barcode from said captured EV; assaying said second DNA barcode using a nucleic acid test; and determining from assaying of said first and second DNA barcodes from said EV whether said disease is present in said subject.

In yet another aspect of the embodiments, the method of assaying further includes: repeating said selectively labeling said extracellular vesicle (EV) from said sample with a further plurality of DNA barcodes such that said EV is labeled with a plurality of distinct capture agents each of which binds to a distinct region of an EV, said first and further plurality of DNA barcodes each being distinguishable from each other; optionally releasing said further plurality of DNA barcodes from said captured EV; assaying said further plurality of DNA barcodes using a multiplex nucleic acid test; and determining a surface protein expression signature from assaying of said plurality of DNA barcodes.

In some embodiments, the method of assaying, further includes: repeating, a plurality of times, the selectively labeling, the selectively capturing, the assaying the DNA barcodes, and the determining of a surface protein expression signature to predict a disease stage and or distinguish between different disease stages. Each selectively capturing uses a capture agent that binds to a portion of the EV that is different from each other capture agent used for the selectively capturing. In one aspects of the embodiments, the method pf assaying includes the releasing the DNA barcodes from the captured EVs before the assaying.

Through a series of bioinformatics and validation processes, the method of the embodiments can identify surface protein expression signatures that distinguish between the stages of a disease. Furthermore, identifying surface protein expression signatures not only has clinical application but also provides novel understanding on the progression of a disease.

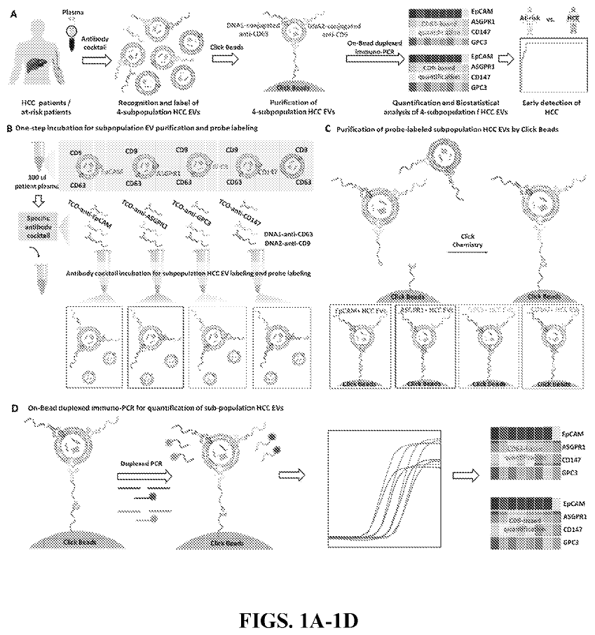

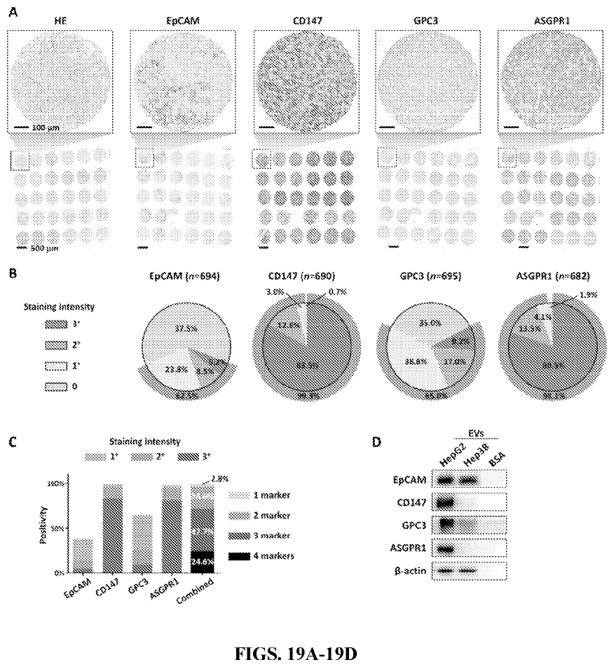

In some embodiments, a streamlined HCC EV Surface Protein Assay (SPA) was designed and developed to distinguish early early-stage HCC from cirrhosis (FIG. 1). Eight subpopulations of HCC EVs were labeling, capturing, and quantifying (i.e., EpCAM⁺ CD63⁺ HCC EVs, CD147⁺ CD63⁺ HCC EVs, GPC3⁺ CD63⁺ HCC EVs, ASGPR1⁺ CD63⁺ HCC EVs, EpCAM⁺ CD9⁺ HCC EVs, CD147⁺ CD9⁺ HCC EVs, GPC3⁺ CD9⁺ HCC EVs, and ASGPR1⁺ CD9⁺ HCC EVs) using the method based on the combined use of click chemistry-mediated EV purification and duplex real-time immuno-PCR. A logistic regression model, named HCC EV ECG score, was established from the resultant HCC EV surface protein signatures (i.e., EpCAM⁺ CD63⁺ HCC EVs, CD147⁺ CD63⁺ HCC EVs, GPC3⁺ CD63⁺ HCC EVs) to distinguish early-stage HCC from cirrhosis. A phase 2 biomarker study was conducted following the International Liver Cancer Association (ILCA) biomarker development guidelines to evaluate the performance of HCC EV ECG score for detecting early-stage HCC. (FIGS. 19-22)

### Assaying

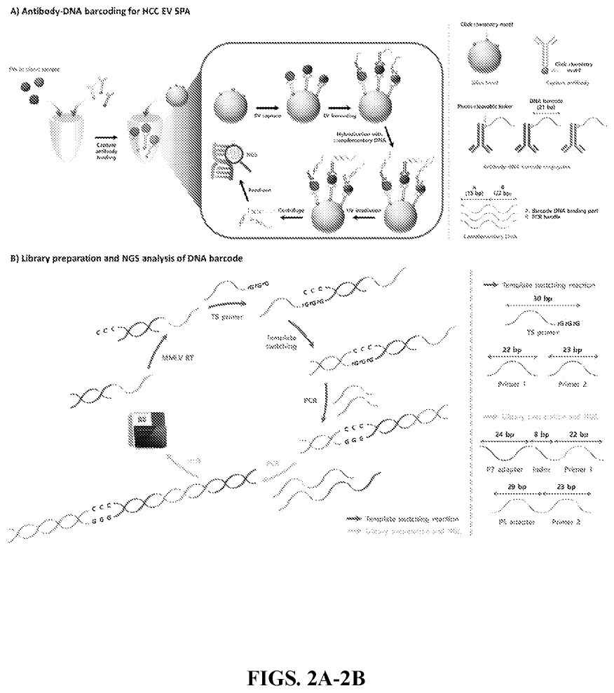

In some embodiments, the assaying can include using a PCR process. In some embodiments, the assaying can include using a multiplex PCR when multiple DNA barcodes have to be quantified. (FIG. 2). In one aspect of the embodiments, the assaying is performed by immune-PCR. In another aspect of the embodiment, the DNA barcodes are quantified by next generation sequencing (NGS) as a downstream readout of our HCC EV surface protein assay (SPA), allowing dramatically improved multiplexing capacity to detect and quantify up to 50-100 surface protein targets on HCC EVs in a single assay. FIG. 2 summarizes the 2-step workflow of this new HCC EV SPA. This is intended to be a non-limiting example for an embodiment directed to HCC. In some embodiments, the assaying can include using a PCR process. In some embodiments, the assaying can include using a nanostring process. These amplification methods can be implemented following protocols all well-known in the art.

To quantify the subpopulations of tumor-derived EVs, an embodiment of the invention EV SPA integrates two powerful technologies: Click Beads for purification of tumor-derived EVs and duplex real-time immuno-PCR for quantification of the purified subpopulations of tumor-derived EVs. Compared with conventional immunoaffinity-based EV capture approaches, Click Beads achieve a more rapid and irreversible purification of HCC EVs by leveraging the click chemistry-reaction between beads and targeted EVs. Notably, the ligation between mTz-grafted capture surface and TCO-grafted EVs not only improved capture efficiency and but also reduced the non-specific EV capture from the background.

An embodiment utilizing multiplex real-time Immuno-PCR, combines the advantages of i) flexibility and robustness of immunoassays and ii) sensitivity of PCR. Immuno-PCR is capable of multiplex detection of several antigens using specific antibodies grafted with DNA barcodes. Real-time immuno-PCR typically exhibits a 10- to 1,000-fold increase in sensitivity compared to an analogous enzyme-amplified immunoassay. As such, the integration of Click Beads and duplex/multiplex real-time immuno-PCR enables tumor-derived EV SPA to sensitively and specifically quantify the tumor-derived EV subpopulations from each individual. Perhaps most important of all, the exemplary embodiment assay only requires a very small amount of plasma (ca. 400 μL) to obtain patients' EV surface protein expression signatures.

In some embodiments, the assaying of multiple barcodes provides surface protein expression signatures distinguishing samples with distinct disease stages. In one aspect of the embodiments, the presence of EpCAM+ CD63+ HCC EVs, CD147+ CD63+ HCC EVs, and GPC3+ CD63+ HCC EVs were observed in patients with HCC compared to those with cirrhosis.

### EXAMPLES

FIG. 1 is provided here to help describe some concepts according to some embodiments of the current invention. However, the general concepts of the current invention are not limited to only labeling EVs with two DNA bar codes. They can be labeled with a single DNA bar code in some embodiments, or more than two bar codes in other embodiments. Examples have been developed to include even up to 50 or 100 DNA bar codes attached to the EVs. However, the general concepts of the current invention are not limited to particular numbers of DNA bar codes. Although HCC is described as a particular example, the general concepts of the current invention are not limited to only HCC.

### Fabrication of Functionalized Click Beads.

Click Beads were prepared by designing a three-step chemical modification procedure (FIG. 4): (i) The silica microbeads with natural hydroxyl (silica MBs, Bangs Laboratories, Inc) were first treated with nitric acid (2 mol/L, Sigma-Aldrich) for 30 min, followed by washing with DI water and ethanol successively. (ii) The resultant silica MBs were then resuspended in (3-aminopropyl) triethoxysilane/ethanol solution (4%, v/v, Sigma-Aldrich) for 45 min to introduce terminal amine groups. Then the amine-terminated silica microbeads (NH₂-modified MBs) were washed with ethanol three times. (iii) To graft mTz motifs, the NH₂-modified MBs was incubated with methyltetrazine-sulfo-NHS Ester (3.8 mM, Click Chemistry Tools) in PBS (200 μL, PH=8.5) for 1 h. The resulting mTz-grafted MBs were washed with DI water three times. After drying under nitrogen flow, the mTz-grafted MBs were stored at 4° C. and retrieved just before use. (APTES, aminopropyltriethoxysilane; DI, deionization; DMSO, dimethyl sulfoxide; MBs, microbeads; mTz, methyltetrazine; NHS, N-hydroxylsuccinimide; PBS, phosphate-buffered saline; PEG, polyethylene glycol; RT, room temperature).

### Preparation of Four TCO-Conjugated HCC-Specific Antibodies

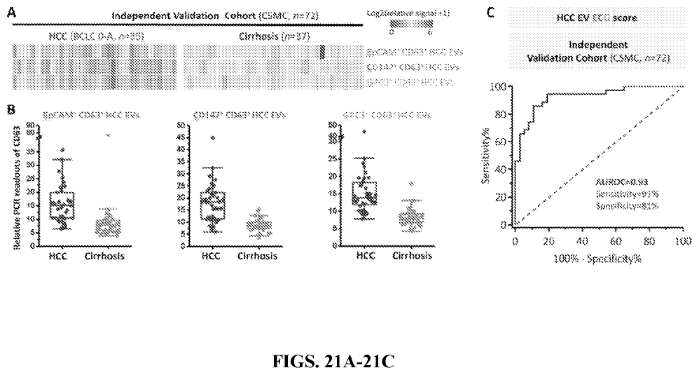

Goat anti-human EpCAM (R&D Systems, Inc., reconstitute at 0.2 mg/mL, dilute 200 to 2,000 times in samples), rabbit anti-human ASGPR1 (LifeSpan BioSciences, Inc., 1 mg/ml, dilute 1,000-10,000 times in samples), sheep anti-human GPC3 (R&D Systems, Inc., reconstitute at 0.2 mg/mL, dilute 200-2,000 times in samples), and goat anti-human CD147 (R&D Systems, Inc., reconstitute at 0.5 mg/mL, dilute 1,000-2,000 times in samples), were incubated with TCO-PEG₄-NHS ester (0.5 mM, Click Chemistry Tools Bioconjugate Technology Company) in PBS at the mole ratio of 1:4, respectively, at room temperature for 30 min. The individual TCO-conjugated antibody was prepared just before use. (FIG. 21) (ASGPR1, Asialoglycoprotein receptor 1; DMSO, dimethyl sulfoxide; EpCAM, epithelial cellular adhesion molecule; GPC3, Glypican 3 Protein; NHS, N-hydroxylsuccinimide; PBS, phosphate-buffered saline; PEG, polyethylene glycol; RT, room temperature; TCO, trans-cyclooctene). The HCC EV SPA relies on the use of different HCC-associated surface protein markers to target and purify subpopulations of HCC EVs in the plasma samples. Recent studies demonstrated the feasibility of selectively characterizing HCC EVs in plasma samples using HCC-associated surface markers, such as EpCAM²³ and ASGPR1.²⁴ In particular, one study²⁴ showed that EVs carrying the surface protein markers, AnnexinV, EpCAM, CD147 and ASGPR1, which are significantly increased in the plasma of HCC patients compared to liver cirrhotic patients and healthy individuals EpCAM, GPC3 and ASGPR1 are HCC-associated surface markers.²⁵ As such, four candidate HCC EV surface protein markers (i.e., EpCAM, ASGPR1, GPC3, and CD147) were selected for validation. Since the surface proteins on tumor-derived EVs could mirror those of the parental tumor cells, we first validated the expression of the four candidate markers using a 708-case HCC TMA, which was generated from archived, resected HCC specimens at UCLA Department of Pathology. The representative TMA H&E staining and IHC staining for the four selected HCC-associated surface protein markers were shown in FIGS. 19A-B summarizes the quantification IHC results of the TMA. Among the four selected markers, CD147 exhibited the highest positivity—of the 708 HCC tumors (excluding those cases without tumor tissues captured on the TMA slides), 576 (83.5%), 88 (12.8%), and 21 (3.0%) were stained as strong (3⁺), moderate (2⁺), and weak (1⁺), respectively. For the commonly used epithelial marker, EpCAM, only 36 (5.2%), 59 (8.5%), and 165 (23.8%) were stained as strong (3⁺), moderate (2⁺), and weak (1⁺), respectively. In terms of the HCC-specific marker GPC3, 64 (9.2%), 118 (17.0%), and 270 (38.8%) were stained as strong (3⁺), moderate (2⁺), and weak (1⁺), respectively. As expected, the majority of HCC tissues are positive for liver-specific marker ASGPR1 with 549 (80.5%), 92 (13.5%), and 28 (4.1%) stained as strong (3⁺), moderate (2⁺), and weak (1⁺), respectively, demonstrating the primary liver origin of the HCC tissues. Overall, 99.7% (701 out of 703) stained positive for at least one marker (24.6% with all four markers positive, 47.7% with any three markers positive, 24.6% with any two markers positive, 2.8% with any one marker positive, FIG. 19C), which underscored the complementary expression of the four surface protein markers in the heterogenous HCC tissues and laid the solid foundation for the use of these HCC-associated antibodies for HCC EV SPA. After identifying, selecting, and conducting TMA validation of the four HCC-associated surface proteins, their presence in two HCC cell line-derived EVs were validated by Western Blotting. As summarized in FIG. 19C), all of the four HCC-associated surface protein markers were detected in HepG2-derived EVs. In Hep3B-derived EVs, both EpCAM and GPC3 were detected.

### Testing the Four HCC-Associated Surface Protein Markers in HCC Cell Line-Derived EVs Using Western Blot

Western blot analysis was performed to quantify the four HCC-associated surface protein markers from the EVs. The collected HepG2- and Hep3B-derived EVs were lysed in an appropriate volume of Radioimmunoprecipitation assay buffer on ice for 30 min followed by centrifugation at 14,000 g for 20 min. The protein concentration was assessed using Qubit assay (Thermo Fisher Scientific). The loading buffers (G-Biosciences) were added and the protein samples were then heated to 95° C. for 5 min. Protein samples were then separated on sodium dodecyl sulfate-polyacrylamide gel electrophoresis, then transferred to polyvinylidene fluoride membranes, blocked in 5% non-fat milk in TBS-T (containing 0.1% Tween-20) for 1 h. The membranes were first incubated with antibodies against β-actin (Sigma-Aldrich Chemie, Steinheim, Germany), EpCAM, CD147, GPC3, and ASGPR1, overnight at 4° C., respectively, and the first antibodies above were dissolved in corresponding blocking solutions, then followed by 1 h incubation with the appropriate secondary antibody. The results were visualized using an enhanced chemiluminescence system.

Conjugation of DNA Barcode onto Antibody and Validation of the Conjugated DNA-Antibody

DNA barcodes having the sequence SEQ ID NO:1 and SEQ ID NO:2 were conjugated onto antibodies for targeting EV markers, CD63 and CD9, respectively (FIG. 6A). (i) Production of N₃-DNA: The DNA barcodes were purchased from IDT and the sequences (FIG. 7, Table 1). N₃-DNA was produced by incubating DNA barcodes with azido-PEG₄-NHS (Click Chemistry Tools) in the molar ratio of 1:20 at room temperature for 4 h. Surplus azido-PEG₄-NHS was removed using Amicon Ultra-0.5 Centrifugal Filter Unit (10K, Millipore). (ii) Preparation of DBCO-antibodies: For anti-CD63 AND anti-CD9, a buffer exchange was performed using 40K Zeba™ spin desalting columns (Thermo Scientific) to 50 mM borate buffered saline (BBS, pH 8.4). Antibodies were incubated for 45 min with NHS-PEG₅-DBCO (Click Chemistry Tools) in the molar ratio of 1:10 at room temperature. Surplus NHS-PEG₅-DBCO was removed using Amicon Ultra-0.5 Centrifugal Filter Unit (50K, Millipore). (iii) Conjugation of DNA-antibodies: SPAAC was used for functionalization of DBCO-antibodies with N₃-DNA for overnight at 4° C. Conjugation of antibody and DNA was performed with a molar ratio of 1:2, 1:4 and 1:10 in PBS to determine functionalization efficiency. Surplus N₃-DNA was removed using Amicon Ultra-0.5 Centrifugal Filter Unit (50 kDa, Millipore) for 2 times. DNA-antibodies were stored in a glycerin/PBS (1:1) solution at −20° C. and retrieved just before use. (FIG. 6B) Gel electrophoresis was used to monitor the conjugation between the DNA-barcode and anti-CD63. Briefly, DNA, DNA-N₃ and DNA-antibody conjugates were run with DNA SafeStain (Lamda Biotech) on a 2% agarose gel (0.5×TBE) and scanned on a gel documentation system. (FIG. 6C) After purification by Amicon centrifugal filter, DNA-conjugated EV-specific antibodies will be characterized by duplex real-time immune-PCR. The linearity of duplex real-time immuno-PCR for detecting (FIG. 6D) DNA1-anti-CD63, R²=0.993, and (FIG. 6E) DNA2-anti-CD9, R²=0.997, respectively. (DBCO, diarylcyclooctyne; EV, extracellular vesicle; IDT, Integrated DNA Technologies; NHS, N-Hydroxysuccinimide; PBS, phosphate buffered saline; PEG, polyethylene glycol; SPAAC, strain-promoted azide-alkyne cycloaddition.

### Optimization of Isolation of HCC EV by Click Beads

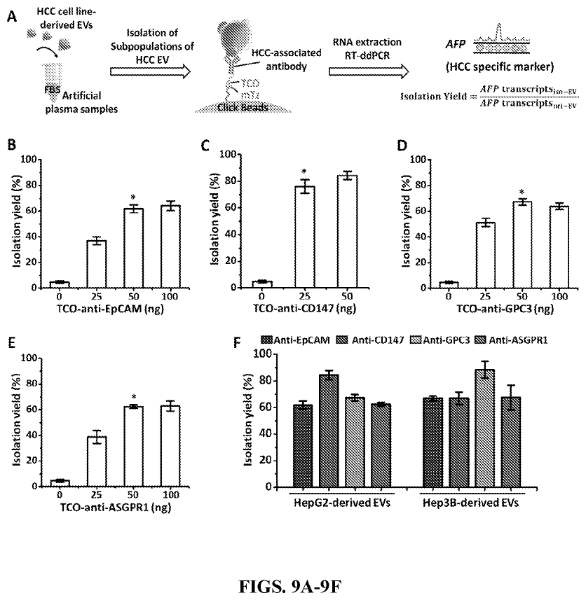

The isolation yield of HCC EV by Click Beads was evaluated by a quantitative method (FIG. 9A). Artificial plasma samples were prepared by spiking 10 μL aliquot of HCC cell line-derived EV pellets into 90 μL healthy donors' plasma. Since AFP transcript was absent in the healthy donor's plasma we tested, the isolation yield of HCC cell line-derived EVs by Click Beads can be calculated from the following equation using RT-ddPCR assay:

$$EV isolation yield = \frac{AFP {transcripts}_{isolated}}{AFP {transcripts}_{original}} \qquad ( 1 )$$

In brief, the original 10 μL aliquot of HCC EVs and the isolated HCC EVs by Click Beads were lysed by 700 μL QIAzol Lysis Reagent (Qiagen), respectively. RNA was extracted using a miRNeasy Micro Kit (Qiagen) following the manufacturer's instructions. The complementary DNA (cDNA) was synthesized using a Maxima H Minus Reverse Transcriptase Kit (Thermo Scientific) following the manufacturer's instructions. cDNA was tested for AFP transcripts using duplex Droplet Digital PCR on a QX200 system (Bio-Rad Laboratories, Inc.) following the manufacturer's instructions and the resulting data were analyzed using the QuantaSoft™ software (Bio-Rad Laboratories, Inc.). The primer and probe for AFP were purchased from Thermo Fisher Scientific (AFP, Hs01040598_m1, 4448489). (FIGS. 9B-E) The isolation yields observed for Click Beads at different amounts of TCO-anti-EpCAM, TCO-anti-CD147, TCO-anti-GPC3, and TCO-anti-ASGPR1, respectively, using the artificial plasma samples spiked with HepG2-derived EVs. Data are presented as means±SD of three independent assays. Asterisks indicate the optimized amount of each TCO-conjugated antibody. (FIG. 9F) The isolation yields observed for Click Beads with the optimized amounts of TCO-conjugated antibodies using the artificial plasma samples spiked with HepG2-derived EVs and Hep3B-derived, respectively.

### Characterization of HepG2-Derived EVs Captured on Click Beads

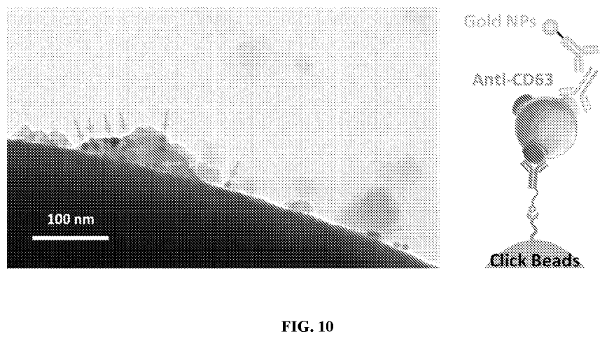

Immunogold staining by anti-CD63 was employed in TEM imaging to verify the identity of HepG2-derived EVs captured on Click Beads. EVs are identified and highlighted with 12-nm gold nanoparticles via anti-CD63. (FIG. 10) Scale bar, 100 nm. Yellow arrows: gold nanoparticles.

### Optimization of HCC EV Surface Protein Assay Using HCC Cell Line-Derived EVs

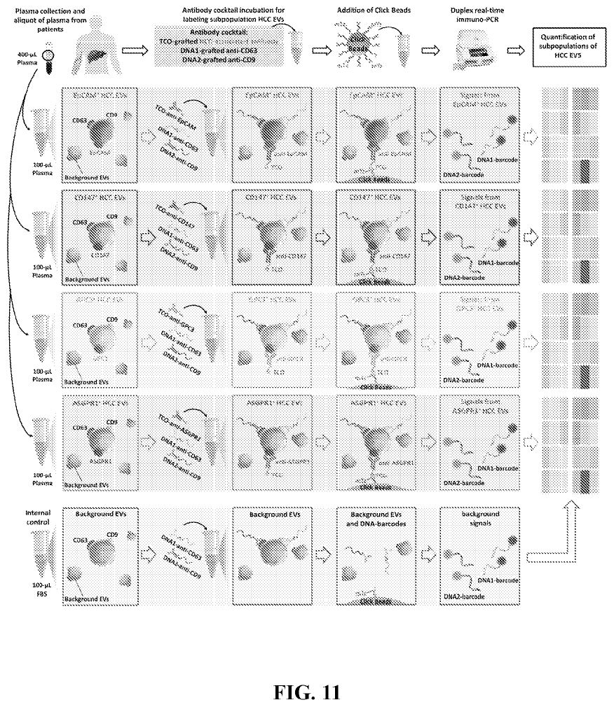

The workflow of HCC EV Surface Protein Assay for quantitative detection of four subpopulations of HCC EVs (EpCAM⁺ HCC EVs as an example) was determined using artificial plasma samples, prepared by spiking 10⁷ HepG2-derived EVs into 100 μL fetal bovine serum (FBS). (FIG. 12A) A duplex real-time immuno-PCR was employed to quantify the copy numbers of DNA1-anti-CD63 labelled on the purified subpopulations of EV samples. (FIG. 12B) Comparison of the relative signals from HCC EV Surface Protein Assay between different blocking conditions. 5% BSA combined with PFBS achieved the best blocking result. (FIG. 12C) DNA1-anti-CD63 has higher signals from HCC EV Surface Protein Assay compared with DNA-control IgG. (FIG. 12D) The relative signal of HCC EV Surface Protein Assay for detecting four subpopulations of HCC EVs. The highest signals were detected in CD147⁺ subpopulations. Good linearity of HCC EV Surface Protein Assay for detecting HepG2 cells-derived EVs were achieved for (FIG. 12E) EpCAM⁺ subpopulation, R²=0.992; (FIG. 12F) CD147⁺ subpopulation, R²=0.989; (G) GPC3⁺ subpopulation, R²=0.999; and (FIG. 12H) ASGPR1⁺ subpopulation, R²=0.980, by DNA1-anti-CD63.

### Validation of Reproducibility of HCC EV Surface Protein Assay Using Clinical Plasma Samples

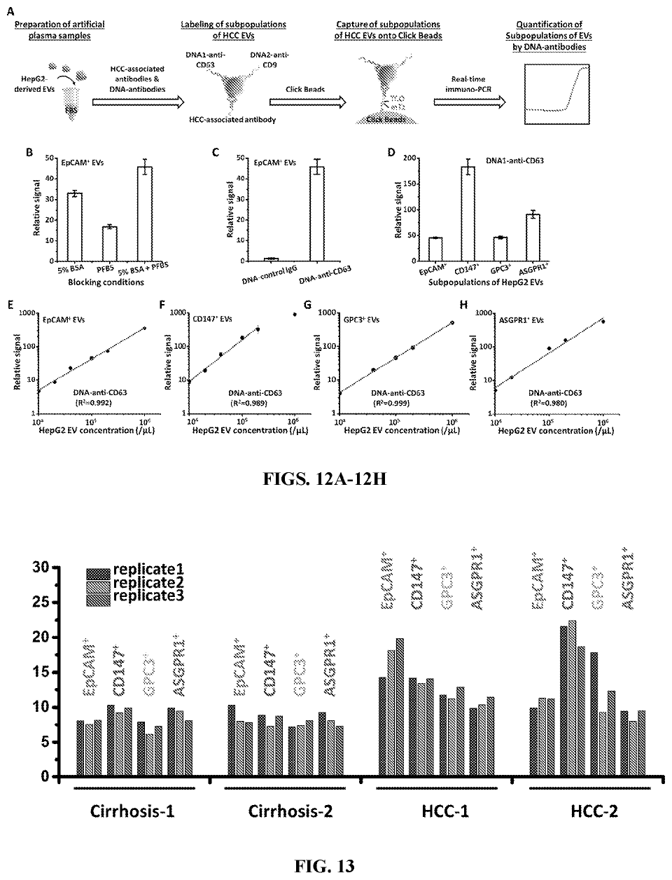

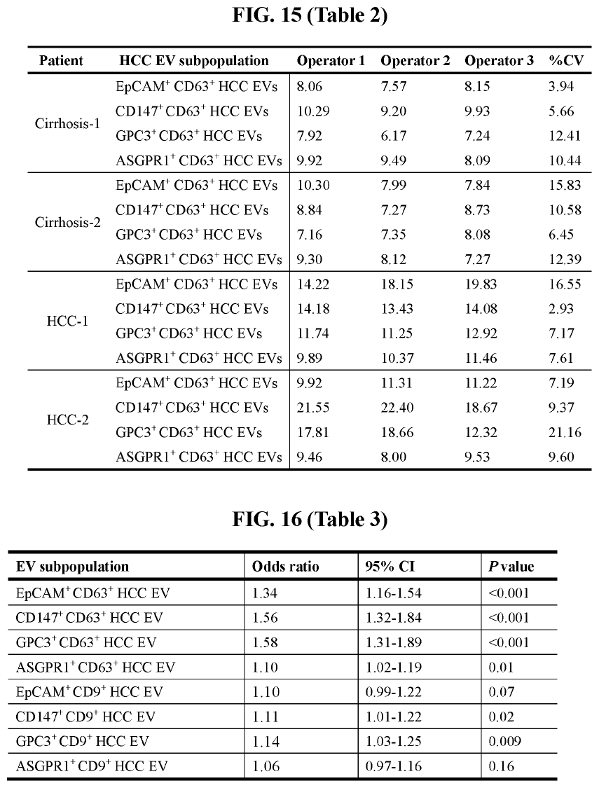

Reproducibility of the quantification of the four HCC EV subpopulations (EpCAM⁺CD63⁺ HCC EVs, CD147⁺ CD63⁺ HCC EVs, GPC3⁺ CD63⁺ HCC EVs, ASGPR1⁺CD63⁺ HCC EVs) isolated from technical triplicate plasma samples from two patients with early-stage HCC and two patients with liver cirrhosis are shown. (FIG. 13) The Coefficients of Variability (CV %) of each subpopulation for each sample is listed in FIG. 15 (Table 2).

ROC Curve of the Eight HCC EV Subpopulations for Detecting Early-Stage HCC from Cirrhosis in the UCLA Training Cohort

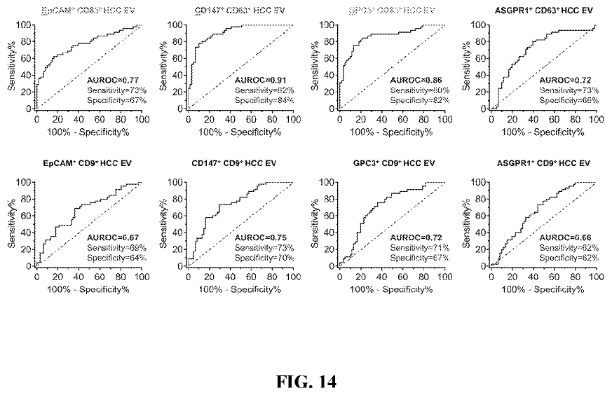

ROC curves of EpCAM⁺ CD63⁺ HCC EVs, CD147⁺ CD63⁺ HCC EVs, GPC3+ CD63⁺ HCC EVs, ASGPR1⁺ CD63⁺ HCC EVs, EpCAM⁺ CD9⁺ HCC EVs, CD147⁺ CD9⁺ HCC EVs, GPC3⁺ CD9⁺ HCC EVs, and ASGPR1 CD9⁺ HCC EVs, respectively, for distinguishing early-stage HCC from cirrhosis in the UCLA training cohort. (FIG. 14)

### Antibody-DNA Barcoding for HCC EV SPA

A total of 100 μL of plasma for each patient was added into an Eppendorf tubes, to which TCO-conjugated HCC-associated antibodies: TCO-anti-EpCAM (25 ng), TCO-anti-CD147 (25 ng), TCO-anti-GPC3 (25 ng), and TCO-anti-ASGPR1 (25 ng) were also added. The plasma samples labeled with TCO-antibodies were then incubated with the Click Beads (0.1 mg) for 30 min for isolation of each subpopulation of EV relying on the Covalent Chemistry-mediated capture of TCO-antibodies on the Click Beads. The Click Beads with immobilized HCC EVs were collected by centrifuge at 10,000 g for 3 min. All plasma samples were subjected to Click Beads with only one freeze-thaw cycle.

After immobilization of HCC EVs onto Click Beads, 10 EV-specific antibody-DNA barcode conjugates, including DNA1-anti-CD63, DNA2-anti-CD9, DNA3-anti-CD81, DNA4-anti-Annexin V, and other DNA barcode-conjugated EV-specific antibodies were incubated with the immobilized HCC EVs. Next, the Click Beads with antibody-DNA barcode conjugates were collected by centrifuge at 10,000 g for 3 min and then the complementary DNA consisting of DNA barcode-binding part and PCR handle which is required for the subsequent NGS library preparation was incubated. A photocleavable linker was incorporated between each pair of DNA barcode and HCC EV-specific antibody, allowing for UV irradiation-triggered photocleavage and recovery of DNA barcodes for quantification by NGS.

### Library Preparation and NGS Analysis of DNA Barcode

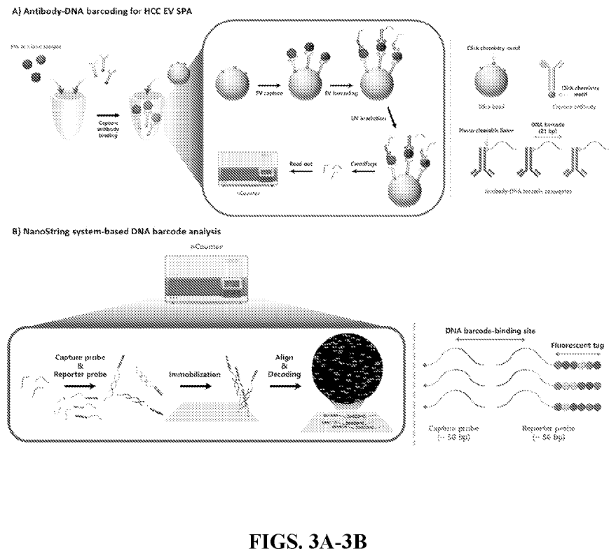

The collected DNA barcodes were then subjected to the library preparation process which begins with the extension of three deoxycytidine triphosphates (dCTPs) from the 3′ end of the complementary DNA by the unique activity of Moloney Murine Leukemia Virus (MMLV) reverse transcriptase (RT). Then, the template switching (TS) primer possessing three guanosine triphosphate (rGTP) at its 3′ end hybridized with the protruding dCTP part of the complementary DNA and template switching reaction occurred, generating the complementary sequence of the TS primer. The subsequent polymerase chain reaction (PCR) with the forward and reverse primer set that bind to the complementary sequences of the TS primer and PCR handle, respectively, amplified the DNA barcode which can be called as region of interest (ROI). The second round of PCR was also executed to further add the P5/P7 adapters and a unique sample index and the final library product was analyzed by using the Illumina NGS system. (FIG. 2) Furthermore, the feasibility of NanoString system as a downstream readout of HCC EV SPA was also evaluated as illustrated in FIG. 3.

### Testing the Four HCC-Associated Surface Protein Markers in HCC Tissue Microarray (TMA)

Hematoxylin and Eosin (H&E) and immunohistochemistry (IHC) staining of the four HCC-associated surface protein markers were performed in a 708-case HCC TMA generated from archived, resected HCC specimens at the University of California, Los Angeles (UCLA). The HCC tissues were fixed in 10% neutral formalin for 24 h and embedded in paraffin according to the standard operating procedure for tissue in the pathology department at UCLA. Serial 5 m-thick tissue microarray slides from formalin-fixed paraffin-embedded blocks were cut and mounted on poly-L-lysine coated glass slides. Standard IHC staining was performed according to a protocol optimized for each antibody, including anti-EpCAM (Cell Signaling, Danvers, MA), anti-CD147 (R&D Systems, Minneapolis, MN), anti-glypican-3 (GPC3; Santa Cruz Biotechnology, Santa Cruz, CA), and anti-asialoglycoprotein receptor (ASGPR; Abcam, Cambridge, UK). All slides were scanned and converted to high resolution digital images with magnification of 40× using the Aperio ScanScope AT high throughput scanning system. The IHC staining results were evaluated using a four-point scale staining intensity (none, 0; weak, 1⁺; moderate, 2⁺; strong, 3⁺) on digital pathology slides by the pathologist (Y. Z.). The HCC tissues were considered to have stained positive if they displayed weak (1⁺), moderate (2⁺) or strong (3⁺) staining to the antibodies tested. (FIG. 19)

### Study Population

For this phase 2 biomarker (case-control) study, a total of 106 participants (45 patients with treatment-naïve early-stage HCC and 61 patients with liver cirrhosis) were enrolled between October 2016-August 2021 at Ronald Reagan University of California, Los Angeles (UCLA) Medical Center as a training cohort to develop and optimize HCC EV SPA for distinguishing early-stage HCC from cirrhosis. A total of 72 participants (35 patients with treatment-naïve early-stage HCC and 37 patients with liver cirrhosis) were enrolled between October 2019-October 2021 at Cedars-Sinai Medical Center (CSMC) as an independent validation cohort. All participants provided written informed consent for this study according to the institutional review board (IRB) protocol #14-000197 at UCLA and IRB protocol #00000066 at CSMC.

HCC was diagnosed according to the American Association for the Study of Liver Diseases clinical practice guideline:⁸ (i) histology or (ii) imaging categorized as Liver Imaging Reporting and Data System 5. Early-stage HCC was defined as BCLC stage 0 or A. Liver cirrhosis was defined according to histology and imaging feature of cirrhosis (nodular surface) or portal hypertension (splenomegaly or portosystemic collaterals). Patients with cirrhosis were ensured the absence of HCC by (i) at least six months of follow-up after blood collection or (ii) a negative contrast-enhanced multiphasic computed tomography or magnetic resonance imaging within two weeks of blood collection. All HCC cases were treatment-naïve at the time of blood collection. Patients were excluded if they had concomitant neoplasms.

Etiologies of underlying liver disease were defined as below. HBV infection was confirmed by positive HBsAg. Hepatitis C virus (HCV) infection was confirmed by (i) HCV RNA or (ii) chronic liver disease with positive anti-HCV. Alcoholic liver disease (ALD) was confirmed by (i) a documented history of ALD or (ii) a significant history of alcohol abuse or alcohol addiction. Nonalcoholic fatty liver disease or nonalcoholic steatohepatitis was diagnosed by (i) histologic or radiologic evidence of fatty infiltration or inflammation without any history of significant alcohol intake (<20 g per day), or (ii) clinical evidence suggesting risk factors for fatty liver disease such as metabolic syndrome without any history of significant alcohol intake.

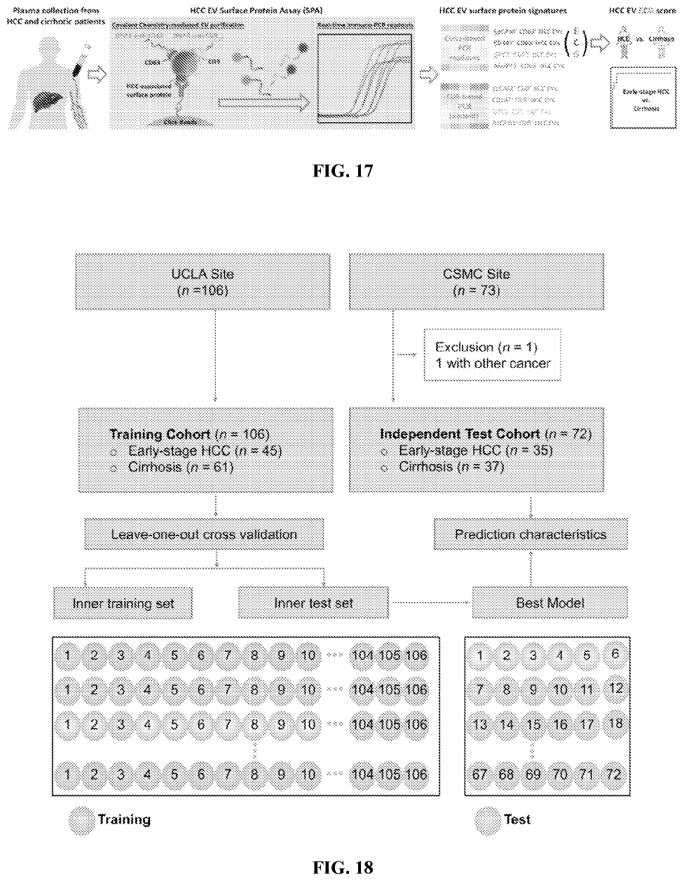

Clinical study design flowchart depicting the recruitment and exclusions from the study cohort. (FIG. 17) Blood samples from 106 and 73 eligible participants were collected from UCLA (training cohort), and CSMC (independent validation cohort), respectively. In brief, logistic regression was utilized in the training cohort (UCLA cohort, n=106) to identify the HCC EV subpopulations significantly associated with early-stage HCC over cirrhosis and establish the logistic regression model (i.e., HCC EV ECG score) for detecting early stage HCC from cirrhosis. Leave-one-out cross validation was applied to estimate the performance of the established HCC EV ECG score in the training cohort. After one participant was excluded due to coexisting with other tumors, external validation of HCC EV ECG score was performed in the independent validation cohort (CSMC cohort, n=72).

### HCC EV Surface Protein Assay (SPA) for Measuring Subpopulations of HCC EVs and Detecting Early-Stage HCC in the UCLA Training Cohort

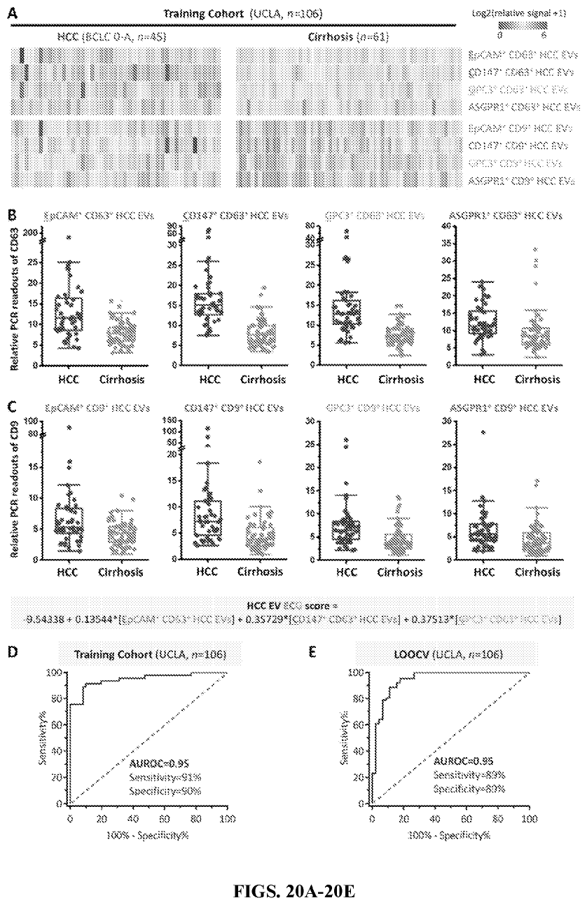

Heatmaps summarize relative duplex realtime immuno-PCR readouts of plasma samples from patients with early-stage HCC (BCLC Stage 0-A, n=45) and patients with liver cirrhosis (n=61). (FIG. 20A) Significantly higher immuno-PCR signals of both (FIG. 20B) CD63+ and (FIG. 20C) CD9+ HCC EV subpopulations were observed in patients with HCC compared to those with cirrhosis. (FIG. 20D) ROC curve of HCC EV ECG score, which was calculated by the signals from EpCAM+ CD63+ HCC EVs, CD147+ CD63+ HCC EVs, and GPC3+ CD63+HCC EVs using HCC EV SPA, for detecting early-stage HCC from cirrhosis in the UCLA training cohort. (FIG. 20E) ROC curve of HCC EV ECG score after leave-one-out cross validation for detecting early-stage HCC from cirrhosis in the UCLA training cohort.

### Blood Sample Processing

Peripheral venous blood samples were collected from participants with written informed consent according to the IRB protocols at UCLA and CSMC. A 10.0-mL ethylenediaminetetraacetic acid vacutainer tube (BD Medical, Fisher Cat. #BD 366643-1) was used for blood sample collection. The whole blood was centrifuged at 530 g for 10 min, and the supernatant was retrieved and centrifuged at 4,600 g for 10 min. The final supernatant was collected as plasma samples and stored in −80° C. refrigerators. All the plasma samples were retrieved just before being subjected to HCC EV SPA.

### General Information for HCC EV SPA

To conduct HCC EV SPA, (i) Click Bead were first produced (i.e., methyltetrazine [mTz]-modified microbeads) according to a chemical modification procedure (FIG. 4), (ii) prepared the four trans-cyclooctene (TCO)-conjugated HCC-associated antibodies using the copper-free Click Chemistry coupling method (FIG. 5), (iii) conjugated DNA barcode onto anti-CD63 and anti-CD9 to target EV markers, followed by validation (FIG. 6). To evaluate the performance of Click Beads for capturing HCC EVs, we prepared HCC cell line-derived EVs according to our previous protocol.¹⁷ Characterization of HepG2-derived EVs (FIG. 8) was performed following the Minimal information for studies of extracellular vesicles (MISEV) 2018²¹ and MISEV 2014²² guidelines issued by the International Society for Extracellular Vesicles. The evaluation of Click Beads for capturing HCC EVs is summarized in FIG. 8 and FIG. 9.

### Characterization of HCC Cell Line-Derived EVs

HCC cell line-derived EVs were prepared according to our previous protocol. Characterization of HepG2-derived EVs was performed following the MISEV 2018³ and MISEV 2014⁴ guidelines issued by the International Society for Extracellular Vesicles. (FIG. 8A) Concentration and size distributions of HepG2-derived EVs (diluted 1:100 in PBS) were measured by nanoparticle tracking analysis using a NanoSight NS300 (Malvern Instruments Ltd, Malvern, UK). The size of the HepG2-derived EVs ranges from 80 to 550 nm. (FIG. 8B) Expression of EV markers (CD63 and Annexin V) on HepG2-derived EVs was analyzed by flow cytometry. (FIG. 8C) Representative TEM image of HepG2-derived EVs in bulk solution before capture with immunogold labeling. EVs are identified and highlighted with gold nanoparticles via anti-CD63. Scale bar, 100 nm. In brief, fixed EVs in PBS were incubated with anti-CD63 (Abcam, mouse, 1:100 dilution) for 30 min. Then, these samples were incubated with nanogolds-conjugated anti-mouse IgG (12 nm, 1:50 dilution) for 1 h. These gold-labeled samples were dropped onto carbon coated copper grids and incubated for 10 min before being wiped off from the grids. After being rinsed 5 times using water, grids were dried for TEM imaging.

### HCC EV SPA for Quantification of Eight Subpopulations of HCC EVs

HCC EV SPA is implemented through a 3-step workflow (FIGS. 1 and 16) and FIGS. 10 and 11). An internal control, fetal bovine serum (FBS), was used instead of plasma samples to measure the background binding of the DNA1-anti-CD63 and DNA2-anti-CD9 to Click Beads.

### Step 1: Sequentially Labeling Each Subpopulation of HCC EVs in Plasma

A total of 400 μL of plasma for each patient were evenly aliquoted into four Eppendorf tubes. In the 1^{st} incubation, these four 100-μL plasma aliquots were incubated with the respective TCO-conjugated HCC-associated antibodies: TCO-anti-EpCAM (50 ng), TCO-anti-CD147 (25 ng), TCO-anti-GPC3 (50 ng), and TCO-anti-ASGPR1 (50 ng), respectively. In the 2^{nd} incubation, these aliquots were cocktailed with DNA-conjugated EV-specific antibodies: DNA1-anti-CD63 and DNA2-anti-CD9, at room temperature for 30 min. No TCO-conjugated HCC-associated antibody was added into the internal control, which was only incubated with DNA1-anti-CD63 and DNA2-anti-CD9.

Step 2: Covalent Chemistry-Mediated Capture of Subpopulations of HCC EVs onto Click Beads

Click Beads¹⁹ were blocked with protein free buffer and then 5% bovine serum albumin (BSA) solution before use. The plasma samples labeled with TCO-antibodies and DNA barcodes were then incubated with the Click Beads (0.1 mg) for 30 min for isolation of each subpopulation of EV. Click Beads with isolated EVs were collected by centrifuge at 10,000 g for 2 min. All plasma samples were subjected to Click Beads underwent only one freeze-thaw cycle. The internal control was processed with the same protocol.

### Step 3: On-Bead Duplex Real-Time Immuno-PCR for Quantifying Each Subpopulation of HCC EVs

Click Beads with isolated EVs were resuspended in 500 μL PBS with 0.2% BSA and then collected by centrifuge at 10,000 g for 2 min. After 3-time washing step with PBS with 0.2% BSA, Click Beads were then washed by PBS for 2 times to wash off unbounded DNA1-anti-CD63 and DNA2-anti-CD9. After 5-time washing steps, 90 μL double-distilled water was added to the sediments of each well and mixed thoroughly. Nine μL of the mixed suspension was loaded into the real-time PCR system (CFX Connect™ Real-Time PCR Detection System, Bio-Rad). The primers and probes for the DNA barcodes were purchased from Integrated DNA Technologies (IDT) and the sequences are listed in FIG. 7 (Table 1). Raw data of real-time PCR runs were evaluated using the corresponding software system (Bio-Rad CFX Manager 3.1, Bio-Rad). The relative signal was calculated using the following equation:

Relative signal=2^{-(Cq(sample)-Cq(internal control))}

### Optimization and Validation of HCC EV SPA Using HepG2-Derived EVs

HepG2-derived EVs were used to optimize and validate HCC EV SPA (FIG. 10). We first blocked Click Beads with 5% BSA, Pierce™ Protein-Free (PBS) Blocking Buffer (PFBS, Thermofisher, #37572), respectively. Then, a DNA-control IgG (anti-PSA, monoclonal mouse IgG1, Abcam) conjugate and DNA-anti-CD63 was used to quantify the EpCAM⁺ subpopulation of HepG2-derived EVs, respectively, to validate the efficacy of DNA-anti-CD63. For the dilution series, concentrations of 10⁴, 105 and 10⁶ particles/μL were spiked into FBS for subpopulation EV isolation with TCO-anti-EpCAM, TCO-anti-ASGPR1, TCO-anti-GPC3 and TCO-anti-CD147, respectively, cocktailed with DNA-anti-CD63 at room temperature for 30 min. As an internal control, FBS samples without spiking HepG2-derived EVs were performed. The samples labeled with TCO-antibodies and DNA-anti-CD63 were then incubated with the EV Click Beads (0.1 mg) for 30 min for the subpopulation EV isolation. Click Beads with isolated EVs were collected by centrifuge at 10,000 g, 2 min. After 5-time washing steps, Click Beads were measured as described above.

### Evaluation of Reproducibility of HCC EV SPA

To test the general applicability of HCC EV SPA, the reproducibility of the HCC EV SPA was evaluated by calculating the percent coefficient of variation (% CV) relative signals. Technical triplicates of two plasma samples from patients with cirrhosis and two plasma samples from patients with HCC were processed with HCC EV SPA by the operators at different timepoints. The evaluation of reproducibility of HCC EV SPA is summarized in FIGS. 11 and 15 (Table 2).

### Statistical Analysis

For descriptive statistics, continuous variables are reported as median and interquartile range, and categorical variables are reported as numbers and percentages. Comparison of continuous variables and categorical variables between groups were done using Mann-Whitney U test and Fisher's exact test or chi-square test, respectively.

In this retrospective phase 2 biomarker (case-control) study, the sample size was calculated for comparing AUCs for the models including HCC EV SPA or serum AFP,¹⁰ using the paired DeLong's test. A sample size of 48 (24 HCC and 24 control) was expected to have 90% power to detect the difference between the AUROCs for our HCC EV SPA versus serum AFP, assuming AUROC=0.93 for our assay, AUROC=0.69 for serum AFP¹⁷ for detecting early-stage HCC, when a correlation between the assays of 0.5 was assumed. The power was obtained for a two-sided test at 0.05 significance level.

To identify the HCC EV subpopulations significantly associated with early-stage HCC over cirrhosis (P<0.05), univariate logistic regression analysis was applied in the training cohort (UCLA cohort; 45 HCC and 61 liver cirrhosis). The HCC EV subpopulations highly associated with HCC at a significance level of 0.005 were included as the final logistic regression model (i.e., HCC EV ECG score) for detecting early-stage HCC from cirrhosis. Youden's index was used to identify the optimal cutoff of HCC EV ECG score. Leave-one-out cross validation was applied to estimate the performance of HCC EV ECG score in the training cohort. External validation of HCC EV ECG score was performed in the independent validation cohort (CSMC cohort; 35 HCC and 37 liver cirrhosis). The overall study design was summarized in FIG. 19. Following the ILCA biomarker development guideline for HCC,²⁰ sensitivity, specificity, and AUROC for HCC EV ECG score to discriminate early-stage HCC from at-risk cirrhosis were estimated in both the training and validation cohorts. The AUROC between the HCC EV ECG score and AFP was compared using the paired DeLong's test.

All statistical analyses were performed using MedCalc software (version 20.015; MedCalc Software Ltd, Ostend, Belgium), GraphPad Prism (version 9.2.0; GraphPad Software, Inc., CA, USA), and R statistical software (version 4.0.2; R Foundation, Vienna, Austria) with two-sided tests and a significance level of 0.05.

Quantification of the Eight Subpopulations of HCC EVs for Distinguishing Early-Stage HCC from at-Risk Liver Cirrhosis in UCLA Cohort

After optimizing HCC EV SPA using artificial samples (FIGS. 9-12) and confirming the reproducibility (FIG. 13 and FIG. 17 (Table 2)), 45 patients with early-stage HCC and 61 at-risk patients with liver cirrhosis were enrolled at UCLA as the training cohort for testing HCC EV SPA. The demographic and clinical characteristics of the UCLA cohort are demonstrated in Table 1. Age, gender, and race/ethnicity were similar between patients with HCC and cirrhotic controls. Approximately three-quarters of patients with HCC (73%) had well-compensated liver disease (Child-Pugh A), compared to nearly half of the cirrhotic controls (44%). Among the HCC patients, 82% had liver cirrhosis. Eighty percent of the HCC patients had BCLC stage A cancer and the rest of them were BCLC stage 0. The optimized HCC EV SPA was utilized to obtain the HCC EV surface protein signatures of the UCLA cohort (FIG. 17). In brief, the identified four HCC-associated antibodies (i.e., anti-EpCAM, anti-CD147, anti-GPC3, and anti-ASGPR1) were applied to purify the EVs and two conjugated DNA-antibodies (i.e., DNA1-anti-CD63 and DNA2-anti-CD9) targeting EV markers were applied to report the presence of EVs. The resulting readouts of eight subpopulations of HCC EVs (i.e., EpCAM CD63⁺ HCC EVs, CD147′ CD63⁺ HCC EVs, GPC3⁺ CD63⁺ HCC EVs, ASGPR1⁺ CD63⁺ HCC EVs, EpCAM CD9⁺ HCC EVs, CD147⁺ CD9⁺ HCC EVs, GPC3⁺ CD9⁺ HCC EVs, and ASGPR1⁺ CD9⁺ HCC EVs) were summarized in FIG. 8A. Overall, significantly higher signals were observed in the HCC group compared to those found in the liver cirrhosis group (P<0.005 in all subpopulations; FIGS. 8B-C).

Development of HCC EV ECG Score for Distinguishing Early-Stage HCC from at-Risk Cirrhosis

To evaluate the ability of the HCC EV surface protein signatures (based on quantification of eight subpopulations HCC EVs) for distinguishing early-stage HCC from at-risk cirrhosis, the UCLA cohort was employed as the training cohort, and the diagnostic performance of each HCC EV subpopulation was summarized in Supplementary FIG. 10. Among the eight HCC EV subpopulations, the AUROCs of CD147⁺ CD63⁺ HCC EVs and GPC3⁺ CD63⁺ HCC EVs are the highest at 0.91 (95% confidence interval [CI]=0.86-0.96) and 0.86 (95% CI=0.79-0.94), respectively. In addition, univariate logistic regression analysis was performed to identify the HCC EV subpopulations significantly associated with early-stage HCC over cirrhosis (FIG. 16, Table 3). Three HCC EV subpopulations highly associated with HCC at a significance level of 0.005, EpCAM CD63⁺ HCC EVs, CD147T CD63⁺ HCC EVs, and GPC3⁺ CD63⁺ HCC EVs, were selected in the final logistic regression model for detecting early-stage HCC from cirrhosis, named HCC EV ECG score.

HCC EV ECG score is defined as:

$$HCC EV ECG score = - 9.54338 + 0 \text{.13544} ⋆ [ \underline{E} {pCAM}^{+} C D6 {3}^{+} HCC EVs ] + 0 \text{.35729} ⋆ [ \underline{C} D 14 {7}^{+} CD {63}^{+} HCC EVs ] + 0 \text{.37513} ⋆ [ \underline{G} PC {3}^{+} CD {63}^{+} HCC EVs$$

This HCC EV ECG score exhibited excellent accuracy for discriminating patients with early-stage HCC from cirrhotic controls in the training cohort with an AUROC of 0.95 (95% CI=0.90-0.99; FIG. 20D). At the optimal cutoff of −0.40, the sensitivity and specificity of HCC EV ECG score for early-stage HCC detection were 91% (95% CI=79%-96%) and 90% (95% CI=80%-95%), respectively. In addition, leave-one-out cross validation of the training cohort confirmed the accuracy of the model with the AUROC of 0.95 (95% CI=0.88-0.98; FIG. 20E), sensitivity of 89% (95% CI=76%-96%), and specificity of 89% (95% CI=78%-95%).

### Performance of HCC EV ECG Score for Detecting Early-Stage HCC in an Independent Validation Cohort

To further validate HCC EV ECG score for detecting early-stage HCC, a total of 35 patients with early-stage HCC and 37 at-risk patients with liver cirrhosis recruited at CSMC were utilized as an independent validation cohort and analyzed. The clinical characteristics of the CSMC cohort are shown in FIG. 7, Table 1 and the HCC EV surface protein signatures of the CMSC cohort were summarized in FIG. 21A. There was no significant difference between CSMC and UCLA cohorts in terms of the patient characteristics. The readouts of EpCAM⁺ CD63⁺ HCC EVs, CD147⁺ CD63⁺ HCC EVs, and GPC3⁺ CD63⁺ HCC EVs were significantly higher in patients with HCC than patients with liver cirrhosis as expected (P<0.001; FIG. 21B).

In the CSMC validation cohort, the AUROC of HCC EV ECG score for detecting early-stage HCC remained excellent as 0.93 (95% CI=0.87-0.99; FIG. 21C). At the cutoff of −0.65, the sensitivity was 94% (95% CI=81%-99%) and the specificity was 81% (95% CI=65%-⁹²%). When setting the cutoff value at −0.40, the same optimal cutoff value identified in the training cohort, HCC EV ECG score still had great accuracy of detecting early-stage HCC with the sensitivity of 91% (95% CI=77%-98%) and the specificity of 81% (95% CI=65%-92%).

### Comparison Between HCC EV ECG Score and Serum AFP for Detecting Early-Stage HCC and Subgroup Analyses

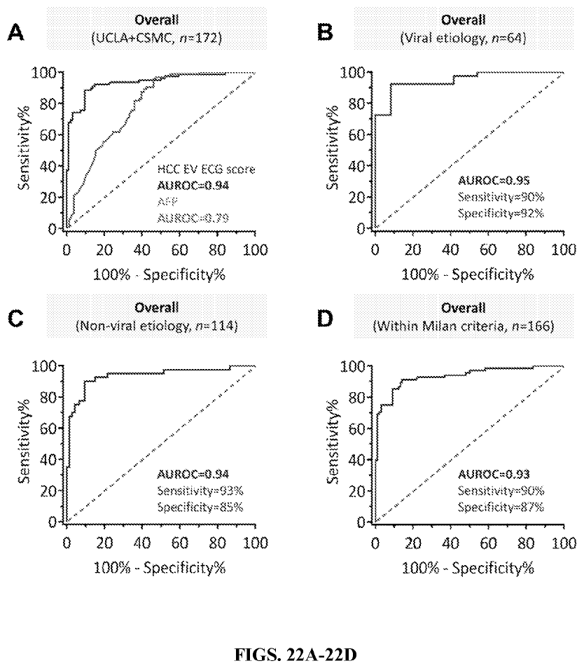

After validating HCC EV ECG score in an independent cohort, we then compared the performance of HCC EV ECG score with serum AFP for detecting early-stage HCC in all the participants in this study (UCLA+CSMC cohorts, n=172). Individuals without serum AFP records (n=4) were excluded in the analyses. As demonstrated in FIG. 22 6A, HCC EV ECG score outperformed serum AFP in distinguishing early-stage HCC from cirrhosis (AUROC=0.94 [95% CI=0.90-0.97] vs. 0.79 [95% CI=0.72-0.86], P<0.001). At the identified cutoff of −0.40, the sensitivity of HCC EV ECG score was 91% (95% CI=82%-96%) and the specificity was 86% (95% CI=78%-⁹²%). For serum AFP, when using the standard cutoff of 20 ng/mL, it had the sensitivity of 45% (95% CI=34%-57%) and the specificity of 98% (95% CI=94%-100%).

The performance of HCC EV ECG score remained excellent in the subpopulations of patients stratified by etiology (viral vs. non-viral) and Milan criteria. Among patients with chronic viral hepatitis (n=64), the AUROC was 0.95 (95% CI=0.90-1.00; FIG. 22B); on the other hand, the AUROC was 0.94 (95% CI=0.88-0.99; FIG. 22C) for patients with non-viral etiology (n=114). When restricting the HCC patients to those with tumor within Milan criteria (n=166), HCC EV ECG score still had excellent performance with AUROC of 0.93 (95% CI=0.89-0.97; FIG. 22D), sensitivity of 90% (95% CI=80%-96%), and a specificity of 87% (95% CI=78%-93%) for HCC detection.

### REFERENCES

1. Sung H, Ferlay J, Siegel R L, et al. Global Cancer Statistics 2020: GLOBOCAN Estimates of Incidence and Mortality Worldwide for 36 Cancers in 185 Countries. CA Cancer J Clin. 2021; 71(3):209-249.
2. Lee Y T, Wang J J, Luu M, et al. State-Level HCC Incidence and Association With Obesity and Physical Activity in the United States. Hepatology. 2021; 74(3):1384-1394.
3. Lee Y T, Wang J J, Luu M, et al. The Mortality and Overall Survival Trends of Primary Liver Cancer in the United States. J Natl Cancer Inst. 2021.
4. Siegel R L, Miller K D, Fuchs H E, Jemal A. Cancer Statistics, 2021. CA Cancer J Clin. 2021; 71(1):7-33.
5. Lee Y T, Wang J J, Luu M, et al. Comparison of Clinical Features and Outcomes Between Intrahepatic Cholangiocarcinoma and Hepatocellular Carcinoma in the United States. Hepatology. 2021; 74(5):2622-2632.
6. Yang J D, Hainaut P, Gores G J, Amadou A, Plymoth A, Roberts L R. A global view of hepatocellular carcinoma: trends, risk, prevention and management. Nat Rev Gastroenterol Hepatol. 2019; 16(10):589-604.
7. De Toni E N, Schlesinger-Raab A, Fuchs M, et al. Age independent survival benefit for patients with hepatocellular carcinoma (HCC) without metastases at diagnosis: a population-based study. Gut. 2020; 69(1):168-176.
8. Marrero J A, Kulik L M, Sirlin C B, et al. Diagnosis, Staging, and Management of Hepatocellular Carcinoma: 2018 Practice Guidance by the American Association for the Study of Liver Diseases. Hepatology. 2018; 68(2):723-750.
9. European Association for the Study of the Liver. Electronic address eee, European Association for the Study of the L. EASL Clinical Practice Guidelines: Management of hepatocellular carcinoma. J Hepatol. 2018; 69(1):182-236.
10. Tzartzeva K, Obi J, Rich N E, et al. Surveillance Imaging and Alpha Fetoprotein for Early Detection of Hepatocellular Carcinoma in Patients With Cirrhosis: A Meta-analysis. Gastroenterology. 2018; 154(6):1706-1718 e1701.
11. Singal A G, Conjeevaram H S, Volk M L, et al. Effectiveness of hepatocellular carcinoma surveillance in patients with cirrhosis. Cancer Epidemiol Biomarkers Prev. 2012; 21(5):793-799.
12. Trinchet J C, Chaffaut C, Bourcier V, et al. Ultrasonographic surveillance of hepatocellular carcinoma in cirrhosis: a randomized trial comparing 3- and 6-month periodicities. Hepatology. 2011; 54(6):1987-1997.
13. Kim S Y, An J, Lim Y S, et al. MRI With Liver-Specific Contrast for Surveillance of Patients With Cirrhosis at High Risk of Hepatocellular Carcinoma. JAMA Oncol. 2017; 3(4):456-463.
14. Lee Y T, Tran B V, Wang J J, et al. The Role of Extracellular Vesicles in Disease Progression and Detection of Hepatocellular Carcinoma. Cancers (Basel). 2021; 13(12).
15. van Niel G, D'Angelo G, Raposo G. Shedding light on the cell biology of extracellular vesicles. Nat Rev Mol Cell Biol. 2018; 19(4):213-228.
16. von Felden J, Garcia-Lezana T, Dogra N, et al. Unannotated small RNA clusters associated with circulating extracellular vesicles detect early stage liver cancer. Gut. 2021.
17. Sun N, Lee Y T, Zhang R Y, et al. Purification of HCC-specific extracellular vesicles on nanosubstrates for early HCC detection by digital scoring. Nat Commun. 2020; 11(1):4489.
18. Dong J, Zhang R Y, Sun N, et al. Coupling Nanostructured Microchips with Covalent Chemistry Enables Purification of Sarcoma-Derived Extracellular Vesicles for Downstream Functional Studies. Adv Funct Mater. 2020; 30(49).
19. Sun N, Tran B V, Peng Z, et al. Coupling lipid labeling and click chemistry enables isolation of extracellular vesicles for noninvasive detection of oncogenic gene alterations. Advanced Science. 2022; In press.
20. Singal A G, Hoshida Y, Pinato D J, et al. International Liver Cancer Association (ILCA) White Paper on Biomarker Development for Hepatocellular Carcinoma. Gastroenterology. 2021; 160(7):2572-2584.
21. Thery C, Witwer K W, Aikawa E, et al. Minimal information for studies of extracellular vesicles 2018 (MISEV2018): a position statement of the International Society for Extracellular Vesicles and update of the MISEV2014 guidelines. J Extracell Vesicles. 2018; 7(1):1535750.
22. Lotvall J, Hill A F, Hochberg F, et al. Minimal experimental requirements for definition of extracellular vesicles and their functions: a position statement from the International Society for Extracellular Vesicles. J Extracell Vesicles. 2014; 3:26913.
23. Willms A, Muller C, Julich H, et al. Tumour-associated circulating microparticles: A novel liquid biopsy tool for screening and therapy monitoring of colorectal carcinoma and other epithelial neoplasia. Oncotarget. 2016; 7(21):30867-30875.
24. Julich-Haertel H, Urban S K, Krawczyk M, et al. Cancer-associated circulating large extracellular vesicles in cholangiocarcinoma and hepatocellular carcinoma. J Hepatol. 2017; 67(2):282-292.
25. Court C M, Hou S, Winograd P, et al. A novel multimarker assay for the phenotypic profiling of circulating tumor cells in hepatocellular carcinoma. Liver Transpl. 2018; 24(7):946-960.
26. Johnson P J, Pirrie S J, Cox T F, et al. The detection of hepatocellular carcinoma using a prospectively developed and validated model based on serological biomarkers. Cancer Epidemiol Biomarkers Prev. 2014; 23(1):144-153.
27. Yang J D, Addissie B D, Mara K C, et al. GALAD Score for Hepatocellular Carcinoma Detection in Comparison with Liver Ultrasound and Proposal of GALADUS Score. Cancer Epidemiol Biomarkers Prev. 2019; 28(3):531-538.
28. Singal A G, Nabihah T, Mehta A, et al. GALAD Demonstrates High Sensitivity for HCC Surveillance in a Cohort of Patients with Cirrhosis. Hepatology. 2021.
29. Ahn J C, Teng P C, Chen P J, et al. Detection of Circulating Tumor Cells and Their Implications as a Biomarker for Diagnosis, Prognostication, and Therapeutic Monitoring in Hepatocellular Carcinoma. Hepatology. 2021; 73(1):422-436.
30. Tran N H, Kisiel J, Roberts L R. Using cell-free DNA for HCC surveillance and prognosis. JHEP Rep. 2021; 3(4):100304.
31. Oussalah A, Rischer S, Bensenane M, et al. Plasma mSEPT9: A Novel Circulating Cell-free DNA-Based Epigenetic Biomarker to Diagnose Hepatocellular Carcinoma. EBioMedicine. 2018; 30:138-147.
32. Chalasani N P, Ramasubramanian T S, Bhattacharya A, et al. A Novel Blood-Based Panel of Methylated DNA and Protein Markers for Detection of Early-Stage Hepatocellular Carcinoma. Clin Gastroenterol Hepatol. 2021; 19(12):2597-2605 e2594.
33. Xu R H, Wei W, Krawczyk M, et al. Circulating tumour DNA methylation markers for diagnosis and prognosis of hepatocellular carcinoma. Nat Mater. 2017; 16(11):1155-1161.
34. Caruso S, Nault J C. A Dive Into the Deep Heterogeneity of Hepatocellular Carcinoma. Gastroenterology. 2019; 157(6):1477-1479.
35. Kolb H C, Finn M G, Sharpless K B. Click Chemistry: Diverse Chemical Function from a Few Good Reactions. Angewandte Chemie International Edition. 2001; 40(11):2004-2021.
36. Karver M R, Weissleder R, Hilderbrand S A. Synthesis and evaluation of a series of 1,2,4,5-tetrazines for bioorthogonal conjugation. Bioconjug Chem. 2011; 22(11):2263-2270.
37. He X, Patfield S A. Immuno-PCR Assay for Sensitive Detection of Proteins in Real Time. Methods Mol Biol. 2015; 1318:139-148.
38. Huang D Q, El-Serag H B, Loomba R. Global epidemiology of NAFLD-related HCC: trends, predictions, risk factors and prevention. Nat Rev Gastroenterol Hepatol. 2021; 18(4):223-238.
39. Sun N, Tran B V, Peng Z, et al. Coupling lipid labeling and click chemistry enables isolation of extracellular vesicles for noninvasive detection of oncogenic gene alterations. Advanced Science 2022; In press.
40. Sun N, Lee Y T, Zhang R Y, et al. Purification of HCC-specific extracellular vesicles on nanosubstrates for early HCC detection by digital scoring. Nat Commun 2020; 11:4489.
41. Thery C, Witwer K W, Aikawa E, et al. Minimal information for studies of extracellular vesicles 2018 (MISEV2018): a position statement of the International Society for Extracellular Vesicles and update of the MISEV2014 guidelines. J Extracell Vesicles 2018; 7:1535750.
42. Lotvall J, Hill A F, Hochberg F, et al. Minimal experimental requirements for definition of extracellular vesicles and their functions: a position statement from the International Society for Extracellular Vesicles. J Extracell Vesicles 2014; 3:26913.

1. A method of assaying for a disease in a subject, comprising:
selectively labeling an extracellular vesicle (EV) from a sample from said subject with a first DNA barcode comprising:
functionalizing a first capture agent for said EV with a first molecule from a first bioorthogonal functional group such that the first capture agent remains attachable to said EV and the first molecule is able to ligate with a second molecule from a second bioorthogonal functional group, the second molecule being complementary to the first molecule,
functionalizing the DNA barcode with a second molecule,
conjugating the functionalized first capture agent with the functionalized DNA barcode to form a barcoded capture agent,
mixing the barcoded capture agent with said sample to form a labeled sample, selectively capturing said EV from said sample from said subject comprising:
functionalizing a second capture agent for said EV with a third molecule from a third bioorthogonal functional group such that the second capture agent remains attachable to said EV and the third molecule is able to ligate with a fourth molecule from a fourth bioorthogonal functional group, the fourth molecule being complementary to the third molecule,
mixing the functionalized second capture agent with the labeled sample such that the functionalized second capture agent binds to said EV forming an activated sample,
functionalizing a capture surface with the fourth molecule, and
depositing at least a portion of the activated sample on at least a portion of the functionalized capture surface to thereby selectively capture said EV by binding of the fourth molecule with the third molecule;
assaying said first DNA barcode by nucleic acid test; and
determining from assaying of said first DNA barcode from said EV whether said disease is present in said subject.

2. A method of assaying for a disease in a subject, comprising:
selectively labeling an extracellular vesicle (EV) from a sample from said subject with a first DNA barcode comprising:
functionalizing a first capture agent for EV with a first molecule from a first bioorthogonal functional group such that the first capture agent remains attachable to EV and the first molecule is able to ligate with a second molecule from a second bioorthogonal functional group, the second molecule being complementary to the first molecule,
functionalizing the DNA barcode with a second molecule,
conjugating the functionalized fist capture agent with the functionalized DNA barcode to form a barcoded capture agent,
functionalizing a second capture agent for said EV with a third molecule from a third bioorthogonal functional group such that the second capture agent remains attachable to said EV and the third molecule is also able to ligate with a fourth molecule from a fourth bioorthogonal functional group, the fourth molecule being complementary to the third molecule,
mixing the barcoded capture agent and the functionalized second capture agent with said sample to form an activated sample,
selectively capturing said EV from said sample from said subject comprising:
functionalizing a capture surface with the fourth molecule, and
depositing at least a portion of the activated sample on at least a portion of the functionalized capture surface to thereby selectively capture said EV by binding of the fourth molecule with the third molecule;
assaying said first DNA barcode by nucleic acid test; and
determining from assaying of said first DNA barcode from said EV whether said disease is present in said subject.

3. The method of assaying according to claim 1, wherein the second capture agent selected for capturing said EV is a tumor-specific capture agent that binds to a surface marker specifically expressed by a tumor-derived EV.

4. The method of assaying according to claim 1, wherein the selectively capturing of an extracellular vesicle (EV) from a sample from a subject is performed prior to the selectively labeling of said EV with a first DNA barcode from said sample from said subject.

5. The method of assaying according to claim 1, further comprises:
repeating said selectively labeling said extracellular vesicle (EV) from said sample with a second DNA barcode such that said EV is labeled with a distinct capture agent binding to a distinct region of an EV, said first and second DNA barcodes being distinguishable from each other;
assaying said second DNA barcode using a nucleic acid test; and
determining from assaying of said first and second DNA barcodes from said EV whether said disease is present in said subject.

6. The method of assaying according to claim 1, further comprises:
repeating said selectively labeling said extracellular vesicle (EV) from said sample with a further plurality of DNA barcodes such that said EV is labeled with a plurality of distinct capture agents each of which bind to a distinct region of an EV, said first and further plurality of DNA barcodes each being distinguishable from each other;
assaying said further plurality of DNA barcodes using a multiplex nucleic acid test; and
determining a surface protein expression signature from assaying of said plurality of DNA barcodes.

7. The method of assaying according to claim 1, further comprises:
repeating, a plurality of times, the selectively labeling, the selectively capturing, the assaying the DNA barcodes, and the determining of a surface protein expression signature to predict a disease stage and or distinguish between different disease stages.

8. The method of assaying according to claim 1, further comprises: releasing the DNA barcode or the plurality of DNA barcodes from the captured EV before assaying.

9. The method of assaying according to claim 8, wherein the surface protein expression signatures distinguishing clinical samples with distinct disease stages.

10. The method of assaying according to claim 9, wherein the expression of EpCAM+ CD63+ on HCC EVs, CD147+ CD63+ on HCC EVs, and GPC3+ CD63+ on HCC EVs is more elevated in patients with HCC compared to those with cirrhosis.

11. The method of assaying according to claim 1, wherein said capture surface is a surface of a bead in a suspension.

12. The method of assaying according to claim 1, wherein said capture surface is a surface of a microfluidic device.

13. The method of assaying according to claim 1, wherein the capture agent that enables the capture of the EV is functionalized with a third bioorthogonal functional group selected from the list consisting of trans-cyclooctene (TCO), alkyne, and a cyclooctyne derivative, and wherein the capture surface is functionalized with a fourth bioorthogonal functional group selected from the list consisting of tetrazine (Tz) and azide.

14. The method of assaying according to any one of claims 1-13, wherein said assaying comprises using a multiplex PCR.

15. The method of assaying according to claim 1, wherein said assaying comprises using a next generation sequencing process.

16. A kit for assaying for a disease in a subject according to claim 1, comprising:
a plurality of capture agents each of which bind to a distinct region of an EV of said subject;
a plurality of molecules with complementary bioorthogonal functional groups;
a plurality of DNA barcodes; and
instructions for labeling said EV with said plurality of DNA barcodes, capturing said EV, assaying said plurality of DNA barcodes, and determining from said assaying whether the disease is present in the subject or the disease stage of the subject.


SEQUENCE LISTING (8 sequences)

SEQ ID NO: 1 - DNA, 85 bp, synthetic construct
  source 1..85: mol_type = other DNA
  misc_feature 1..85: note = Barcode DNA 1 (for CD63)
cgagggaagtctgatacagcggcaaggatcacgttaggccgatgtgccaatacgccgacaattgccataccgtcgtccactgctt

SEQ ID NO: 2 - DNA, 73 bp, synthetic construct
  source 1..73: mol_type = other DNA
  misc_feature 1..73: note = Barcode DNA 2 (for CD9)
tggtctgtggtgctgtattgagcggtgcgatcacgacatcacgacatcacgacccaaactgagtagccttccc

SEQ ID NO: 3 - DNA, 20 bp, synthetic construct
  source 1..20: mol_type = other DNA
  misc_feature 1..20: note = Forward Primer
aagtctgatacagcggcaag

SEQ ID NO: 4 - DNA, 18 bp, synthetic construct
  source 1..18: mol_type = other DNA
  misc_feature 1..18: note = Reverse Primer
gcagtggacgacggtatg

SEQ ID NO: 5 - DNA, 24 bp, synthetic construct
  source 1..24: mol_type = other DNA
  misc_feature 1..24: note = Probe (FAM)
tcacgttaggccgatgtgccaata

SEQ ID NO: 6 - DNA, 20 bp, synthetic construct
  source 1..20: mol_type = other DNA
  misc_feature 1..20: note = Forward Primer
tggtctgtggtgctgtattg

SEQ ID NO: 7 - DNA, 20 bp, synthetic construct
  source 1..20: mol_type = other DNA
  misc_feature 1..20: note = Reverse Primer
gggaaggctactcagtttgg

SEQ ID NO: 8 - DNA, 24 bp, synthetic construct
  source 1..24: mol_type = other DNA
  misc_feature 1..24: note = Probe (HEX)
tgcgatcacgacatcacgacatca